(19) United States
(12) Patent Application Publication
(10) Pub. No.: US 2023/0416791 A1
(43) Pub. Date: Dec. 28, 2023

(54) METHOD TO PRODUCE BRANCHED-CHAIN POLYHYDROXYALKANOATES AND BRANCHED-CHAIN 3-HYDROXYACIDS FROM GLUCOSE

(71) Applicant: Alliance for Sustainable Energy, LLC, Golden, CO (US)

(72) Inventors: Gregg Tyler BECKHAM, Golden, CO (US); Gayle Joann BENTLEY, Edgewater, CO (US); Christopher W. JOHNSON, Denver, CO (US); Kevin Joseph McNAUGHT, Westminster, CO (US)

(21) Appl. No.: 18/123,943

(22) Filed: Mar. 20, 2023

Related U.S. Application Data
(60) Provisional application No. 63/321,207, filed on Mar. 18, 2022.
(60) Provisional application No. 63/479,918, filed on Jan. 13, 2023.

(51) Int. Cl. C12P 7/625; C12N 9/10

(52) U.S. Cl. CPC C12P 7/625; C12N 9/1029; C12Y 203/01041; C12Y 203/0118

(57) ABSTRACT

Disclosed herein are methods and compositions that enable the production of branched-chain polyhydroxyalkanoates, branched-chain 3-hydroxyacids (BCHA) (PHA monomers), and branched-chain fatty acids (BCFA) in Pseudomonas putida KT2440. The branched-chain molecules enabled by this platform enable novel chemistries that are not accessible via the existing paradigm which is limited to straight-chain molecules.

Further disclosed herein are methods and compositions for deciphering the mechanisms of bacterial fatty acid biosynthesis is crucial for both the engineering of bacterial hosts to produce fatty acid-derived molecules and the development of new antibiotics.

### CROSS-REFERENCE TO RELATED APPLICATIONS

This application claims the benefit of U.S. Patent Application No. 63/321,207 filed on 18 Mar. 2022 and U.S. Patent Application No. 63/479,918 filed on 13 Jan. 2023, both of which are incorporated by reference herein in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### SEQUENCE LISTING

This application contains a Sequence Listing which has been submitted electronically and is hereby incorporated by reference in its entirety. The XML document as filed herewith was originally created on 18 Sep. 2023. The XML document as filed herewith is named NREL 21-63.xml, is 86 kilobytes in size and is submitted with the instant application.

### BACKGROUND

Deciphering the mechanisms of bacterial fatty acid biosynthesis is crucial for both the engineering of bacterial hosts to produce fatty acid-derived molecules and the development of new antibiotics. However, gaps in our understanding of the initiation of fatty acid biosynthesis remain. Here, we demonstrate that the industrially relevant microbe Pseudomonas putida KT2440 contains three distinct pathways to initiate fatty acid biosynthesis. The first two routes employ conventional β-ketoacyl-ACP synthase III enzymes, FabH1 and FabH2, that accept short- and medium-chain-length acyl-CoAs, respectively. The third route utilizes a malonyl-ACP decarboxylase enzyme, MadB. A combination of exhaustive in vivo alanine-scanning mutagenesis, in vitro biochemical characterization, X-ray crystallography, and computational modelling elucidate the presumptive mechanism of malonyl-ACP decarboxylation via MadB. Given that functional homologs of MadB are widespread throughout domain Bacteria, this ubiquitous alternative fatty acid initiation pathway provides new opportunities to target a range of biotechnology and biomedical applications.

Polyhydroxyalkanoates (PHAs) are chiral biopolymers that are naturally accumulated in some bacterial organisms as a mechanism of carbon storage. Due to their natural biodegradability and structural properties, significant investments have been made to develop commercial materials based on these biopolymers. PHAs have been successfully commercialized to replace some high-density polyethylene and other hard plastics. However, despite significant research, a PHA replacement for low density polyethylene (LDPE), the most highly consumed plastic, has been unsuccessful due to the inability to mimic LDPE physical properties. Branched-chain PHAs have the promise to more precisely mimic the properties of LDPE, but their production has never been demonstrated from glucose.

### SUMMARY

In an aspect, disclosed herein are non-naturally occurring P. putida sp. comprising a non-naturally occurring gene encoding for malonyl-ACP decarboxylase wherein the gene has greater than 70% sequence identity to the nucleotide sequence of PP_0262 from P. putida KT 2440.

In an aspect, disclosed herein are genetically engineered Pseudomonas useful for the degradation of polyhydroxyalkanoates.

In an aspect, disclosed herein is a non-naturally occurring Pseudomonas sp. comprising a non-naturally occurring gene encoding for malonyl-ACP decarboxylase (MadB) wherein the gene (madB) has greater than 70% sequence identity to the nucleotide sequence of PP_0262 from P. putida KT 2440. In an embodiment, the non-naturally occurring Pseudomonas sp. is Pseudomonas sp. is P. putida KT 2440.

In an aspect, disclosed herein is a method for initiating fatty acid biosynthesis in P. putida sp. comprising overexpressing a non-naturally occurring gene encoding for malonyl-ACP decarboxylase (MadB) wherein the gene (madB) has greater than 70% sequence identity to the nucleotide sequence of PP_0262 from P. putida KT 2440.

In an aspect, disclosed herein is a non-naturally occurring Pseudomonas sp. useful for the production of branched-chain polyhydroxyalkanoates, branched-chain 3-hydroxyacids (BCHA) (PHA monomers), and branched-chain fatty acids (BCFA) comprising an overexpressed gene selected from the group consisting of sfabH2, BKD (lpdV-bkdAA-bkd-AB-bkdB), lplA, fadR, phaC, phaG, alkK, acc, alsS-ilvCD, and leuAmodBCD. In an embodiment, the non-naturally occurring Pseudomonas sp. is P. putida KT 2440. In an embodiment, the gene is sfabH2. In an embodiment, the gene is BKD (lpdV-bkdAA-bkd-AB-bkdB). In an embodiment, the gene is lplA. In an embodiment, the gene is fadR. In an embodiment, the gene is phaC. In an embodiment, the gene is phaG. In an embodiment, the gene is alkK. In an embodiment, the gene is acc. In an embodiment, the gene is alsS-ilvCD. In an embodiment, the gene is leuAmodBCD.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts a proposed fatty acid biosynthesis pathway in P. putida KT2440. Box 102 of FIG. 1 depicts that initiation culminates either with the synthesis of a β-ketoacyl-ACP species (β-acetoacetyl-ACP via FabH1 or β-keto-decanoyl-ACP via FabH2), or generation of acetyl-ACP from decarboxylation of malonyl-ACP by MadB. Box 104 of FIG. 1 depicts that each catalytic cycle extends the growing acyl-ACP chain by two carbons donated from malonyl-ACP. Box 106 of FIG. 1 depicts that fatty acid biosynthesis is terminated when one of the elongation intermediates is consumed to generate a fatty acid-derived product. The production of medium-chain-length polyhydroxyalkanoate from (R)-β-hydroxyacyl-ACP and the liberation of free fatty acid by a thioesterase (TE) are shown as examples.

FIGS. 2A, 2B, 2C, and 2D depict supporting information for P. putida containing two nonessential KASIII homologs with different acyl-CoA preferences. FIG. 2A depicts the genomic context of identified KASIII homologs FabH1 (PP_4379) and FabH2 (PP_4545). FIG. 2B depicts the frowth curves of wild-type P. putida and indicated deletion mutants (n=5) grown in a modified M9 media with glucose as the carbon source. FIG. 2C depicts the maximum specific growth rates of wild-type E. coli and ΔfabH E. coli strains transformed with pBTL-2 plasmid bearing indicated genes (n=5). FIG. 2D depicts the enzymatic reactions (I, II, III) demonstrating the preferred acyl-CoA donor of EcFabH, FabH1, and FabH2 as detected by LC-MS.

FIGS. 3A, 3B, 3C, 3D, and 3E depict that MadB is implicated in fatty acid initiation. FIG. 3A depicts a schematic outlining the production and utilization of a P. putida genomic library to screen for novel fatty acid initiation factors. FIG. 3B depicts genomic fragments contained within indicated plasmids that rescued the E. coli ΔfabH colony size defect. Dotted line reflects ambiguity in fragment junction. FIG. 3C depicts the maximum specific growth rates of wild-type E. coli and ΔfabH E. coli strains transformed with pBTL-2 plasmid bearing indicated genes (n=5). FIG. 3D depicts the quantification of cell size of wild-type E. coli, ΔEcfabH, and ΔEcfabH+madB dividing cells (n=8, 13, 5, respectively). FIG. 3E depicts the maximum specific growth rates of wild-type E. coli and indicated mutant strains at 37° C. (n=5).

FIGS. 4A, 4B and 4C depict that MadB and FabH1 are the main fatty acid initiation factors in P. putida. FIG. 4A depicts growth curves of wild-type P. putida and indicated deletion mutants (n=5). FIG. 4B depicts growth curves of wild-type P. putida and ΔmadB ΔfabH1 supplemented with the indicated length straight-chain fatty acids at 100 μg/mL (n=5). FIG. 4C depicts serial dilutions of wild-type P. putida and ΔfabH1 ΔfabH2 ΔmadB bearing a temperature-sensitive plasmid with madB grown at 25° C. and 37° C. on LB agar plates for 16 hrs.

FIGS. 5A, 5B, 5C and 5D depict that madB encodes a malonyl-ACP decarboxylase. FIG. 5A depicts enzymatic reactions (I, II, III, IV) demonstrating the conversion of malonyl-ACP to acetyl-ACP by addition of MadB as detected by LC-MS. FIG. 5B depicts maximum specific growth rates of wild-type and ΔfabH E. coli strains transformed with pBTL-2 plasmid bearing madB variants (n=5). Dashed gray horizontal line indicates maximum specific growth rate of ΔfabH E. coli expressing madB, above which might indicate rescue. FIG. 5C depicts the active site of MadB with malonyl-CoA computationally docked. Dashed lines indicate the hydrogen bonding network and the asterisk represent residue from the subunit B. FIG. 5D depicts a docking model of a MadB dimer interacting with two molecules of malonyl-ACP shown in spacefill.

FIGS. 6A, 6B, and 6C depict a proposed molecular mechanism of MadB-catalyzed decarboxylation of malonyl-ACP. FIG. 6A depicts a proposed decarboxylation mechanism. The R-group of the substrate is an ACP-bound pantetheine arm; however, a methyl group was used for the DFT calculation. FIG. 6B depicts a free energy profile along the computed reaction coordinates. DFT-optimized key intermediates and transition states along the MadB reaction coordinates following (as depicted in FIG. 6C) the lowest energy reaction C3 carbonyl-stabilized pathway.

FIGS. 7A, 7B, and 7C depict distribution and in vivo activity of Mad homologs within the domain Bacteria. (A) Protein domain structure of MadB and EcMadA. Green box indicates the HDF Malonyl-ACP Decarboxylase (MAD) domain, previously referred to as YiiD_C (PF09500). Orange box indicates the GNAT domain (PF13673). (B) Count data for MadB/MadA-like proteins found in Proteobacteria species and beyond. (C) Maximum specific growth rates of wild-type E. coli and ΔfabH E. coli strains transformed with pBTL-2 plasmid bearing homologs from indicated species (n=5). Dashed gray horizontal line indicates maximum specific growth rate of ΔfabH E. coli, above which might indicate rescue. Media supplemented with 0.1 mM IPTG. Tree indicates relatedness of the protein sequences from each species generated by MUSCLE.

FIG. 8 depicts a pathway for branched chained polyhydroxyalkanoates (BCPHA) accumulation in P. putida KT2440. Branched-chain α-ketoacids can be derived from native branched-chain amino acid biosynthesis (module 1). A branched-chain-α-ketoacid dehydrogenase (BKD) activates branched-chain α-ketoacids to branched-chain acyl-CoA starters (BCCSs) which then enter Fatty Acid Synthase II (FASII) biosynthesis by condensation with a malonyl-ACP by a branched-chain-specific FabH (module 2). Branched-chain acyl-ACPs will undergo elongation in FASII until they are released to a 3-hydroxyacyl-CoA by PhaG. Branched 3-hydroxyacyl-CoAs are then polymerized by PhaC (module 3).

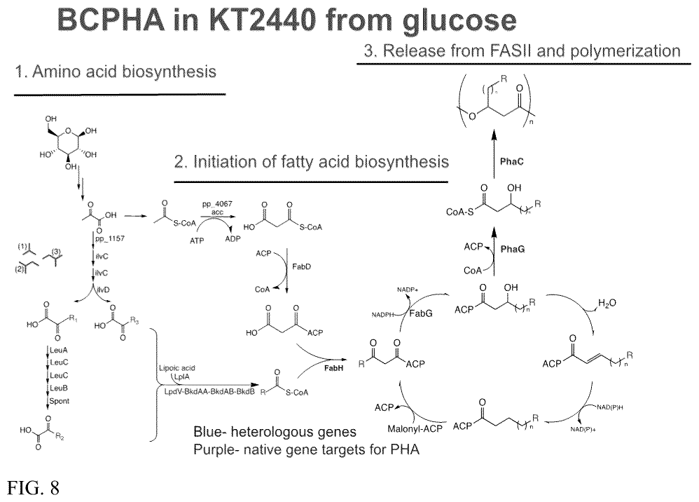

### DETAILED DESCRIPTION

Pseudomonas putida KT2440 contains three distinct pathways to initiate fatty acid biosynthesis. The first two routes employ conventional β-ketoacyl-ACP synthase III enzymes, FabH1 and FabH2, that accept short- and medium-chain-length acyl-CoAs, respectively. The third route utilizes a malonyl-ACP decarboxylase enzyme, MadB. A combination of exhaustive in vivo alanine-scanning mutagenesis, and in vitro biochemical characterization, elucidate the presumptive mechanism of malonyl-ACP decarboxylation via MadB. Given that functional homologs of MadB are widespread throughout domain Bacteria, this ubiquitous alternative fatty acid initiation pathway provides new opportunities to target a range of biotechnology and biomedical applications.

Polyhydroxyalkanoates (PHAs) are biodegradable polymers many bacteria produce and accumulate as intracellular granules for the purpose of carbon storage. The composition of the PHA synthesized by a given bacterium depends on the monomers it produces and the specificity of its PHA synthase, the enzyme that polymerizes these monomers, both of which can be manipulated using synthetic biology. PHAs with different compositions have different physical properties that make them suitable for different applications. Owing to this and their inherent biodegradability, PHAs have been of commercial interest for decades.

Prior PHA properties have not met the criteria to replace low density polyethylene. By incorporating a branched-chain PHA, we may improve the physical properties of PHAs to serve as direct, biodegradable replacements for LDPE.

The ability to use BCPHA as a LDPE replacement was determined based on the prior characterization of BCPHA by others. However, others first synthesized the branched-chain fatty acid, then fed this fatty acid to the cell, at which point, the cell polymerized the supplemented BCFA into BCPHA. BCFA are incredibly expensive to purchase and difficult to chemically synthesize. Disclosed herein are at least one efficient biological route that can convert a biomass-derived feedstock into branched-chain PHAs, branched-chain 3-hydroxyacids, and BCFA. Prior to methods and compositions disclosed herein, the production of branched-chain 3-hydroxyacids and BCPHA has never been demonstrated from a sugar carbon source.

Pipelines to enable the production of branched-chain polyhydroxyalkanoates, branched-chain 3-hydroxyacids (BCHA) (PHA monomers), and branched-chain fatty acids (BCFA) in Pseudomonas putida KT2440 have been developed. All of the branched-chain molecules enabled by this platform enable novel chemistries that are not accessible via the existing paradigm which is limited to straight-chain molecules. These pathways may also be applied to other production hosts, as the pathways are modular. Pseudomonas putida KT2440 has been extensively validated as a host capable of producing high dry cell weight percentages of polyhydroxyalkanoates, with a variety of functional groups, including branched-chains. Disclosed herein is an organism engineered to accumulate branched-chain 3-hydroxy-acyl-CoAs, which can be incorporated into polymerized BCPHAs by the native PHA synthase PhaC. The 3-hydroxyacid monomers can also be produced as monomers without the presence of PhaC.

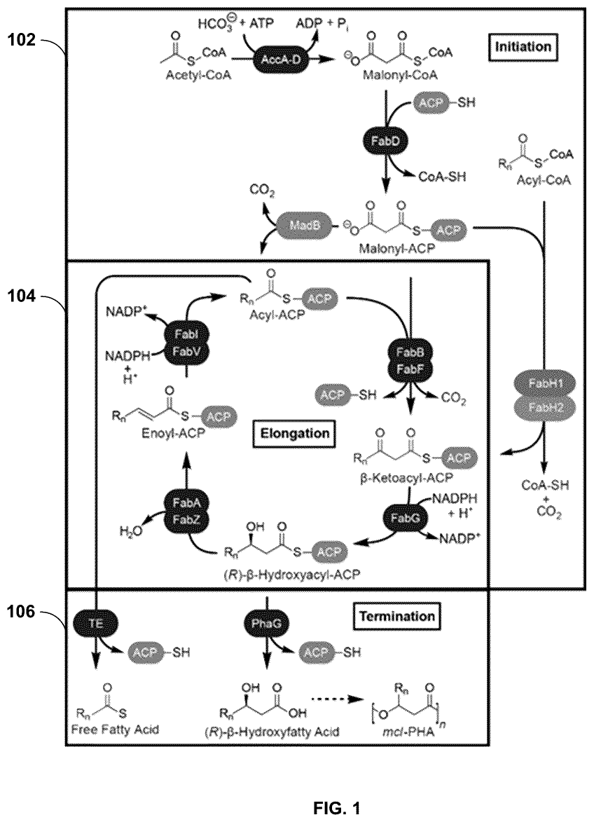

Fatty acid biosynthesis in P. putida KT2440 can be used for the production of BCPHA by incorporating a branched-chain production module. Branched-chain-acyl-ACPs undergoing elongation in the Fatty Acid Synthase II complex can be diverted to PHA biosynthesis by expression of the native PhaG, which transacylates the branched 3-hydroxyacyl-ACP to a branched-chain 3-hydroxyacl-CoA which is then the substrate for the PHA polymerase PhaC (FIG. 1). Previous BCPHA production demonstrates that native PHA machinery is compatible with branched-chain substrates, albeit from synthesized branched-chain fatty acid feedstocks. This would be the first example of the native production of BCPHA from a renewable feedstock. In addition to expression of the branched-chain production module, the strain will be engineered for enhanced overall PHA production. Overexpression of phaG, phaC and acetyl-CoA carboxylase (encoded by acc) have been shown to improve PHA accumulation and fatty acid biosynthesis, respectively. Deletion of phaF prevents negative regulation of phaC. Deletion of phaZ prevents BCPHA depolymerization and subsequent utilization as a carbon source6.

Genes required for expression are described in Table 1, organized by module pictured in FIG. 1.

TABLE 1
Steady-state kinetic parameters from ITC analyses for MadB
and select variants for malonyl-CoA decarboxylation.

| | K_{cat} | K_{M} | k_{cat}/K_{M} |
|---|---|---|---|
| MadB variant | (s⁻¹) | (mM) | (s⁻¹ · M⁻¹) |
| wild-type | 1.4 ± 0.1 | 12 ± 1 | 120 ± 20 |
| Asn45Ala | 0.06 ± 0.01 | 6.9 ± 0.5 | 9 ± 2 |
| His46Ala | 0.45 ± 0.01 | 5.7 ± 0.2 | 79 ± 4 |

In the upstream Module 1, in addition to overexpression of the isoleucine and leucine biosynthetic genes under constitutive synthetic promoters, a feedback resistant mutant of LeuA (leuAmut, G562D) will be expressed to avoid negative autoregulation of the pathway. Previous work has additionally determined that expression of the branched-chain alpha-ketoacid dehydrogenase limits its own lipoylation and the lipoylation of other 2-oxoacid dehydrogenases, particularly pyruvate dehydrogenase. Expression of the lipoyl ligase A (lplA) can complement a lipoylation defect and concomitantly enhance branched-chain fatty acid production. In order to maximize BCPHA production, additional genes must be deleted to remove negative regulation of PHA biosynthesis, prevent beta-oxidation, and to direct fatty acid biosynthesis strictly towards branched-chain structures. Base strains AG2228 and AG2162 engineered for general PHA accumulation will be modified for BCPHA accumulation. These base strains are designed to limit depolymerization and negative regulation of PHA biosynthesis, along with overexpression of the PHA biosynthetic genes. The full genotypes for each strain are listed in Table 2.

The same modules can be applied to produce branched-chain fatty acids (BCFA), as well as branched-chain 3-hydroxyacid monomers (BCHA). BCPHAs and fatty acids are derived from fatty acid biosynthesis, so the same modules can be applied to produce both BCFA and BCHA.

To that end, to achieve FFA production in P. putida, a thioesterase can be expressed which releases the nascent fatty acyl-ACP from the fatty acid elongation machinery, forming a free fatty acid. We have chosen the highly efficient thioesterase from E. coli, tesA, ensuring that this protein will remain cytosolic where its activity is required. Further, we tested KT2440 expressing 'tesA (GB138), and two strains optimized for PHA production also transformed with the 'tesA (GB136: P. putida KT2440 ΔphaZ ΔfadB1-fadA1 ΔfadB2-fadA2-fadE2 pBTL2-'tesA and GB137: P. putida KT2440 ΔphaZ ΔfadB1-fadA1ΔhdhA::pTac-phaG-alkK-phaC1-phaC2 ΔfadB2-fadA2-fadE2 pBTL2-'tesA).

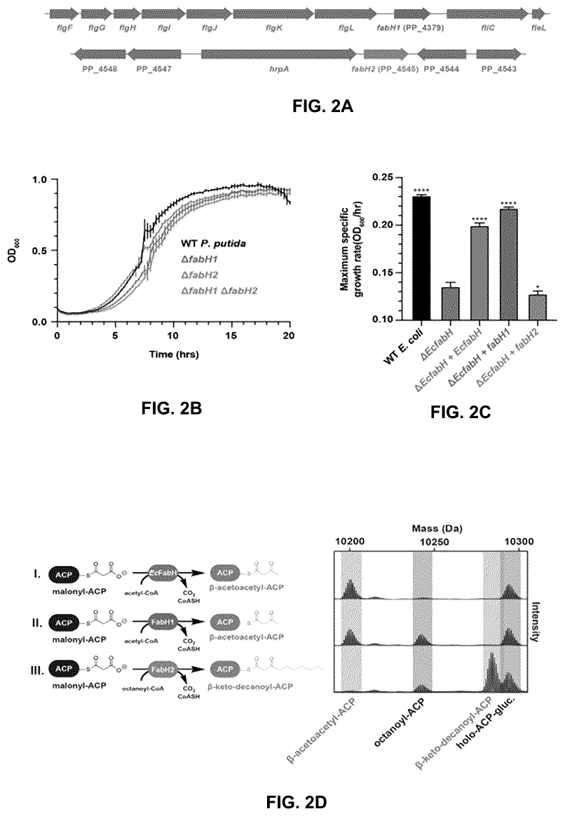

Expression of a thioesterase in the above strains of P. putida KT2440 enabled the production of FFA, with strain GB136 producing the highest titer of FFA, under a nitrogen replete condition (FIG. 2). In general, each strain produced more FFA in higher nitrogen medium, reflecting our expectation that FFA will compete with native PHA production. Despite this potential competition, expression of a thioesterase can indeed hydrolyze the acyl-ACP to FFA, at titers reaching 350 mg/L. This information provides a baseline for future strain optimization, including providing branched-chain precursors.

BCHA production can be achieved by expression of a medium-chain-specific thioesterase, tesB, as well as overexpression of PhaZ, the PHA depolymerase, in addition to the deletion of PhaC and PhaG, the PHA polymerases. In combination with the branched-chain upstream modules, these modifications enable the production of BCHA, monomers with significant downstream platform chemical potential. Again, the branched-chain enables novel chemistries as the terminal methylation dramatically alters the physical properties of the monomer, as compared to a straight-chain monomer.

TABLE 2
Overexpressed genes for BCPHA production

| Overexpressed Gene | Function | Species Origin | Module |
|---|---|---|---|
| ₛfabH2 | Initiates FASII with branched-chain | Bacillus subtilis | 2 |
| | specificity | | |
| BKD (lpdV-bkdAA- | Activates branched-chain alpha- | Bacillus subtilis | 2 |
| bkd-AB-bkdB) | ketoacids to CoA | | |
| lplA | Ligates lipoic acid to the E2 subunit | Escherichia coli | 2 |
| | of BKD | | |
| fadR | Fatty acid transcriptional regulator | Escherichia coli | 2 |
| phaC, phaG, alkK | PHA synthase, 3-hydroxyacyl- | P. putida | 3 |
| | ACP:CoA transacylase, Medium- | | |
| | chain fatty acyl-CoA ligase | | |
| acc | Acetyl-CoA carboxylase | P. putida | 1 |
| alsS-ilvCD | Generates 3-methyl-2-oxobytyric | B. subtilis, E. coli | 1 |
| | acid precursor from pyruvate | | |
| leuA^{mod}BCD | Generates 4-methyl-2-oxopentanoic | E. coli | 1 |
| | acid from 3-methyl-2-oxobytyric | | |
| | acid | | |

TABLE 3
Base PHA strains for BCPHA strain construction.

| Strain | Genotype |
|---|---|
| AG2162 | P. putida KT2440 ΔphaZ(PP_5004) ΔfadB1-fadA1(PP_2136- |
| | 2137) ΔhdhA(PP_0545)::pTac-phaG(PP_1408)-alkK(PP_0763)- |
| | phaC1(PP_5003)-phaC2(PP_5005) ΔfadB2-fadA2-fadE2 |
| | (PP_2214-2217). |
| AG2228 | AG2228: P. putida KT2440 ΔphaZ(PP_5004) ΔfadB1-fadA1 |
| | (PP_2136-2137) ΔfadB2-fadA2-fadE2 (PP_2214-2217) |

In an embodiment, disclosed herein are methods and compositions of matter comprising non-naturally occurring and naturally occurring P. putida KT2440 that harbors three pathways for the initiation of fatty acid biosynthesis. Two of these pathways utilize β-ketoacyl-ACP synthase III enzymes, FabH1 and FabH2, that are specific to short and medium-chain length acyl-CoAs, respectively. The third pathway proceeds through the decarboxylation of malonyl-ACP to acetyl-ACP as catalyzed by a hotdog fold protein, MadB. Of the three pathways, FabH1 and MadB are the primary fatty acid initiation factors in P. putida KT2440. DFT calculation suggests that MadB catalyzes its reaction by stabilizing the C3-carbonyl moiety of the substrate.

Functional homologs of MadB are prevalent in the domain Bacteria and may serve as a novel target for biotechnology and biomedical applications.

The production of membrane lipids is an essential process for all known forms of cellular life. Except for Archaea, which synthesize membrane lipids from isoprenoids, bacteria, plants, and animals utilize fatty acids for membrane production. In animals, the suite of reactions required for fatty acid biosynthesis are performed by a type I fatty acid synthesis (FASI) pathway. Canonical FASI consists of a single polypeptide chain that folds to form several catalytic domains capable of iteratively elongating the covalently attached acyl chain. In contrast, most bacteria have a type II fatty acid synthesis (FASII) pathway, which is given by a distributed system of enzymes that catalyze each step of this biosynthetic process. The modular nature of FASII, in part, underlies the diversity of fatty-acid derived products bacteria can generate.

FASII can be broadly partitioned into three sets of reactions: initiation, elongation, and termination (FIG. 1). FASII initiation culminates in the production of a β-ketoacyl group covalently attached to acyl carrier protein (ACP) (box 102 of FIG. 1). In the case of Escherichia coli, EcFabH (β-ketoacyl-ACP synthase III (KASIII)) catalyzes the Claisen condensation of acetyl-CoA with malonyl-ACP to produce the β-acetoacetyl-ACP primer. Moreover, the KASIII condensation reaction may also be catalyzed by a variant of the KASI/II domain containing protein which is exemplified by FabY from P. aeruginosa. Notably, homologs of EcFabH from other species have also been shown to accept alternative acyl-CoAs, including branched-chain acyl-CoAs and medium-chain-length acyl-CoAs. Regardless of their identity, these varied β-ketoacyl-ACP primers all subsequently proceed through a series of reactions that elongate their chain length by two carbons in each catalytic cycle (box 104 of FIG. 1). The resultant acyl-ACPs terminate the fatty acid elongation cycle when used as substrates in the production of membrane phospholipids or other fatty acid-derived products, such as polyhydroxyalkanoates (box 106 of FIG. 1). Modulating different stages of fatty acid biosynthesis can alter the profile of fatty acid-derived products generated and in extreme cases can potentially lead to.

More recently an alternate KAS-independent FASII initiation pathway was discovered in E. coli in the form of malonyl-ACP decarboxylase (Mad, also referred to as YiiD). The Mad system was first identified in ΔfabH strains which exhibiting growth and colony morphology defects, yet remained viable, thereby suggesting the presence of an FabH-independent initiation pathway. The catalytic domain of Mad is housed by a hot-dog fold (HDF) domain that may exist as a fusion with a Gcn5-related N-acetyl transferase (GNAT) domain (MadA), such as that discovered in E. coli, or as a stand-alone protein (MadB). Ultimately, the Mad system produces acetyl-ACP that can enter the elongation step via condensing enzymes such as of FabB/F.

Despite discoveries in model bacterial species such as E. coli and P. aeruginosa, there are significant gaps in our understanding of fatty acid biosynthesis in non-traditional model organisms. For example, although numerous publications reported the engineering of the industrially relevant microbe Pseudomonas putida KT2440 to produce polyhydroxyalkanoates, a fatty acid-derived product, a comprehensive, mechanistic understanding of how fatty acids are produced in this species is lacking and thus preventing rational engineering of the system. To that end, here we elucidate the three pathways for the initiation of fatty acid biosynthesis in P. putida. We find that P. putida harbors two KASIII enzymes, FabH1 (PP_4379) and FabH2 (PP_4545), that accept short- and medium-chain-length acyl-CoAs, respectively. We also demonstrate that ΔfabH1 ΔfabH2 double mutants are not only viable but exhibit wild type-like growth rates due to an undescribed gene, PP_0262, that we identified independently in a forward-genetic screen. Biochemical and phylogenetic characterization of PP_0262 established that it is a MadB enzyme. Whole-protein alanine-scanning mutagenesis, isothermal titration calorimetry (ITC), X-ray crystallography, and structural and computational studies elucidate the putative mechanism of action of MadB. In addition, we demonstrate that functional homologs of MadB are present in a variety of bacteria, highlighting the widespread nature of this enzyme in fatty acid biosynthesis.

Material & Methods

Plasmid construction. Primers used in this study are described in Table 4. Plasmids used in this study are described in Table 5. We used Phusion® High-Fidelity PCR Master Mix (NEB) for all polymerase chain reactions. NEBuilder HiFi DNA Assembly Master Mix (NEB) was used for plasmid construction followed by transformation into chemically competent NEB 5-α F′Iq E. coli. Golden Gate assembly was utilized for the generation of CRISPR guide RNA-expressing plasmids. Transformants were selected on plates made with LB Broth with agar (Miller; Sigma-Aldrich) supplemented with kanamycin (5011 g/mL), carbenicillin (10011 g/mL), apramycin (5011 g/mL), or spectinomycin (5011 g/mL) where appropriate and grown overnight at 37° C. Resulting constructs were confirmed by Sanger sequencing (GENEWIZ, Inc.).

TABLE 4
Primers disclosed herein.

| SEQ ID | | | |
|---|---|---|---|
| NO | Primer | Sequence (5′-3′) | Description |
| SEQ ID | oKM_0001 | acgcctcacacaggaaacagctatgacTACGCAGCGCTCTGG | 5′ forward primer to |
| NO: 1 | | AAAAG | generate ΔPP_4379 variant |
| | | | of pK18sB |
| SEQ ID | oKM_0002 | gcttttaggggctgactgggatcatctggcttcccaatgaaaacagcc | 5′ reverse primer to |
| NO: 2 | | | generate ΔPP_4379 variant |
| | | | of pK18sB |
| SEQ ID | oKM_0003 | ggctgttttcattgggaagccagatgatcccagtcagcccctaaaagc | 3′ forward primer to |
| NO: 3 | | | generate ΔPP_4379 variant |
| | | | of pK18sB |
| SEQ ID | oKM_0004 | acgttgtaaaacgacggccagtGCCATCGAGCAGATTACGG | 3′ reverse primer to |
| NO: 4 | | C | generate ΔPP_4379 variant |
| | | | of pK18sB |
| SEQ ID | oKM_0005 | CACCAGCGACACCGAGTATT | Diagnostic: Forward primer |
| NO: 5 | | | to verify deletion of PP_4379 |
| SEQ ID | oKM_0006 | CACCGATGGTCGGCTTATCA | Diagnostic: Reverse primer |
| NO: 6 | | | to verify deletion of PP_4379 |
| SEQ ID | oKM_0007 | acgcctcacacaggaaacagctatgacTCCTTGAAGGCGAAC | 5′ forward primer to |
| NO: 7 | | AACCG | generate ΔPP_4545 variant |
| | | | of pK18sB |
| SEQ ID | oKM_0010 | acgttgtaaaacgacggccagtCAGCCATCCTGCAGTCGTT | 3′ reverse primer to |
| NO: 8 | | C | generate ΔPP_4545 variant |
| | | | of pK18sB |
| SEQ ID | oKM_0011 | CTGAGCTACCACTTCGAGCC | Diagnostic: Forward primer |
| NO: 9 | | | to verify deletion of PP_4545 |
| SEQ ID | oKM_0012 | ACACCCTGAAGGACAACGTC | Diagnostic: Reverse primer |
| NO: 10 | | | to verify deletion of PP_4545 |
| SEQ ID | oKM_0049 | AGGGAGAAAGGCGGACAGGTATCC | Diagnostic: Forward primer |
| NO: 11 | | | to verify cloning insertion |
| | | | into pK18sB |
| SEQ ID | oKM_0050 | CGGAGAACCTGCGTGCAATCCATC | Diagnostic: Reverse primer |
| NO: 12 | | | to verify cloning insertion |
| | | | into pK18sB |
| SEQ ID | oKM_0053 | atagtcctgtcgggtttc | Sequencing: Forward primer |
| NO: 13 | | | to sequence pK18sB |
| | | | insertions |
| SEQ ID | oKM_0054 | ccatcttgttcaatcatgcg | Sequencing: Reverse primer |
| NO: 14 | | | to sequence pK18sB |
| | | | insertions |
| SEQ ID | oKM_0055 | acgcctcacacaggaaacagctatgacAGTTCGAATTTGCCAA | 5′ forward primer to |
| NO: 15 | | CGGC | generate ΔPP_0262 variant |
| | | | of pK18sB |
| SEQ ID | oKM_0056 | aaggccgcgttagtcgtactataacggttgtcctcttgatcgtcatg | 5′ reverse primer to |
| NO: 16 | | | generate ΔPP_0262 variant |
| | | | of pK18sB |
| SEQ ID | oKM_0057 | catgacgatcaagaggacaaccgttatagtacgactaacgcggcctt | 3′ forward primer to |
| NO: 17 | | | generate ΔPP_0262 variant |
| | | | of pK18sB |
| SEQ ID | oKM_0058 | acgttgtaaaacgacggccagtATTGCCGGCAGTGAAGAGT | 3′ reverse primer to |
| NO: 18 | | T | generate ΔPP_0262 variant |
| | | | of pK18sB |
| SEQ ID | oKM_0059 | GTTGATTCTGGGCGAAACCG | Diagnostic: Forward primer |
| NO: 19 | | | to verify deletion of PP_0262 |
| SEQ ID | oKM_0060 | CCGGGAGCTCAAGGAAGAAG | Diagnostic: Reverse primer |
| NO: 20 | | | to verify deletion of PP_0262 |
| SEQ ID | oKM_0061 | gcgcgggggtaaacgtatg | Sequencing: Primer to |
| NO: 21 | | | sequence ΔPP_4545 variant |
| | | | of pK18sB |
| SEQ ID | oKM_0062 | ctgggtgctgcctacctgat | Sequencing: Primer to |
| NO: 22 | | | sequence ΔPP_4545 variant |
| | | | of pK18sB |
| SEQ ID | oKM_0063 | cgtaacaggcgcctgatgaatcaggtcgttcctctggtcaaaggc | 5′ reverse primer to |
| NO: 23 | | | generate ΔPP_4545 variant |
| | | | of pK18sB |
| SEQ ID | oKM_0064 | gcctttgaccagaggaacgacctgattcatcaggcgcctgttacg | 3′ forward primer to |
| NO: 24 | | | generate ΔPP_4545 variant |
| | | | of pK18sB |
| SEQ ID | oKM_0082 | taatacgactcactatagg | Sequencing: T7 promoter |
| NO: 25 | | | primer |
| SEQ ID | oKM_0083 | gctagttattgctcagcgg | Sequencing: T7 terminator |
| NO: 26 | | | primer |
| SEQ ID | oKM_0108 | TAGTATTCTCTGGATTCACGCGGT | CRISPR-Cas9 gRNA |
| NO: 27 | | | forward oligo for E. coli fabH |
| SEQ ID | oKM_0109 | AAACaccgcgtgaatccagagaat | CRISPR-Cas9 gRNA |
| NO: 28 | | | reverse oligo for E. coli fabH |
| SEQ ID | oKM_0110 | GATGTGCTGGTTTGTGACGG | 5′ forward primer to |
| NO: 29 | | | generate ΔfabH via |
| | | | CRISPR-Cas9 |
| SEQ ID | oKM_0113 | GACGCATCATCCAGACCGAT | 3′ reverse primer to |
| NO: 30 | | | generate ΔfabH via |
| | | | CRISPR-Cas9 |
| SEQ ID | oKM_0122 | agcgagtcagtgagcgag | Sequencing: Primer to |
| NO: 31 | | | sequence gRNA insertion |
| | | | into pTargetF |
| SEQ ID | oKM_0154 | ccgccctcagaaaaactcatcg | Reverse primer to generate |
| NO: 32 | | | pBTL-2 lacking BamHI site |
| SEQ ID | oKM_0155 | cgatgagtttttctgagggcggtccccctcaagtcaaaagcc | Forward primer to generate |
| NO: 33 | | | pBTL-2 lacking BamHI site |
| SEQ ID | oKM_0156 | ttcacactctagagggatccgtcaacgaattcaagcttgatatcattcag | Forward primer to generate |
| NO: 34 | | | pBTL-2 with BamHI site in |
| | | | MCS |
| SEQ ID | oKM_0157 | ggatccctctagagtgtgaaattgttatccgct | Reverse primer to generate |
| NO: 35 | | | pBTL-2 with BamHI site in |
| | | | MCS |
| SEQ ID | oKM_0164 | aacaatttcacactctagaggttttcattgggaagccagaatgatt | Forward primer to generate |
| NO: 36 | | | pBTL-2::PP_4379 |
| SEQ ID | oKM_0165 | ggctcgtcctgaatgatatctcagtcgcggtacaggatgg | Reverse primer to generate |
| NO: 37 | | | pBTL-2::PP_4379 |
| SEQ ID | oKM_0166 | aacaatttcacactctagagctttgaccagaggaacgaccgt | Forward primer to generate |
| NO: 38 | | | pBTL-2::PP_4545 |
| SEQ ID | oKM_0167 | ggctcgtcctgaatgatatctcagcgcttgcgcagaat | Reverse primer to generate |
| NO: 39 | | | pBTL-2::PP_4545 |
| SEQ ID | oKM_0168 | aacaatttcacactctagagcgaaaagtgactgagcgtacatgtat | Forward primer to generate |
| NO: 40 | | | pBTL-2::fabH |
| SEQ ID | oKM_0169 | ggctcgtcctgaatgatatcctagaaacgaaccagcgcgga | Reverse primer to generate |
| NO: 41 | | | pBTL-2::fabH |
| SEQ ID | oKM_0170 | aacaatttcacactctagaggtgacattctggttgcaggtatgag | Forward primer to generate |
| NO: 42 | | | pBTL-2::fabY |
| SEQ ID | oKM_0171 | ggctcgtcctgaatgatatcctactcttcttogttcccgccc | Reverse primer to generate |
| NO: 43 | | | pBTL-2::fabY |
| SEQ ID | oKM_0172 | ctttctgctatggaggtcaggtatgatt | Sequencing: Forward primer |
| NO: 44 | | | to sequence pBTL-2 inserts |
| SEQ ID | oKM_0173 | ttgtccagcagggttgtccac | Sequencing: Reverse primer |
| NO: 45 | | | to sequence pBTL-2 inserts |
| SEQ ID | oKM_0174 | ctgcctcggtgagttttctcc | Sequencing: Primer to |
| NO: 46 | | | sequence deletion of BamHI |
| | | | site in pBTL-2 |
| SEQ ID | oKM_0175 | gcaaattgcgtcatgttttaatccttatcctagtacgctcagtcacttttcggt | 5′ reverse primer to |
| NO: 47 | | | generate ΔfabH via |
| | | | CRISPR-Cas9 |
| SEQ ID | oKM_0176 | accgaaaagtgactgagcgtactaggataaggattaaaacatgacgcaa | 3′ forward primer to |
| NO: 48 | | tttgc | generate ΔfabH via |
| | | | CRISPR-Cas9 |
| SEQ ID | oKM_0180 | ctctagagtgtgaaattgttatccgct | Reverse primer to linearize |
| NO: 49 | | | pBTL-2 |
| SEQ ID | oKM_0181 | gatatcattcaggacgagcctcag | Forward primer to linearize |
| NO: 50 | | | pBTL-2 |
| SEQ ID | oKM_0240 | aacaatttcacactctagaggatcaagaggacaaccgttaatgagca | Forward primer to generate |
| NO: 51 | | | pBTL-2::PP_0262 |
| SEQ ID | oKM_0241 | ggctcgtcctgaatgatatcctagcggtgcagaacgtactg | Reverse primer to generate |
| NO: 52 | | | pBTL-2::PP_0262 |
| SEQ ID | oKM_0256 | AGAAGGAGATATACATATGagcaccgatagccagtac | Forward primer to clone |
| NO: 53 | | | PP_0262 into pET-21b(+) |
| | | | digested with NdeI/BamHI |
| SEQ ID | oKM_0257 | AGCTCGAATTCGGATCCCTAGCGGTGCAGAACGTA | Reverse primer to clone |
| NO: 54 | | CTG | PP_0262 into pET-21b(+) |
| | | | digested with NdeI/BamHI |
| SEQ ID | oKM_0259 | gtacgctcagtcacttttcggt | 5′ reverse primer for fabH |
| NO: 55 | | | replacement via CRISPR- |
| | | | Cas9 |
| SEQ ID | oKM_0260 | gataaggattaaaacatgacgcaatttgc | 3′ forward primer for fabH |
| NO: 56 | | | replacement via CRISPR- |
| | | | Cas9 |
| SEQ ID | oKM_0265 | accgaaaagtgactgagcgtacatgagcaccgatagccagtacc | Forward primer for PP_0262 |
| NO: 57 | | | replacement of fabH via |
| | | | CRISPR-Cas9 |
| SEQ ID | oKM_0266 | gcaaattgcgtcatgttttaatccttatcctagcggtgcagaacgtactg | Reverse primer for PP_0262 |
| NO: 58 | | | replacement of fabH via |
| | | | CRISPR-Cas9 |
| SEQ ID | oKM_0278 | ACCATCATCACCACAGCCAGagcactatcgaagaacgcgt | Forward primer to clone E. |
| NO: 59 | | | coli acpP into pETDuet-1 |
| | | | digested with BamHI/EcoRI |
| SEQ ID | oKM_0279 | AGGCGCGCCGAGCTCGttacgcctggtggccgtt | Reverse primer to clone E. |
| NO: 60 | | | coli acpP into pETDuet-1 |
| | | | digested with BamHI/EcoRI |
| SEQ ID | oKM_0280 | ATAAGAAGGAGATATACATATGgcaatattaggtttaggcac | Forward primer to clone E. |
| NO: 61 | | gga | coli acpS into pETDuet-1 |
| | | | digested with NdeI/XhoI |
| SEQ ID | oKM_0281 | GCGGTTTCTTTACCAGACttaactttcaataattaccgtggcaca | Reverse primer to clone E. |
| NO: 62 | | agc | coli acpS into pETDuet-1 |
| | | | digested with NdeI/XhoI |
| SEQ ID | oKM_0282 | GATTATGCGGCCGTGTACAA | Sequencing: Reverse primer |
| NO: 63 | | | to sequence acpP insertion |
| | | | into pETDuet-1 |
| SEQ ID | oKM_0283 | TTGTACACGGCCGCATAATC | Sequencing: Forward primer |
| NO: 64 | | | to sequence acpS insertion |
| | | | into pETDuet-1 |
| SEQ ID | oKM_0284 | AGACCACGGTGAGCAATGAC | Sequencing: Forward primer |
| NO: 65 | | | 1 to sequence |
| | | | pKM133/pKM138 |
| SEQ ID | oKM_0285 | attgcagtcctcttcggacagc | Sequencing: Forward primer |
| NO: 66 | | | 2 to sequence |
| | | | pKM133/pKM138 |
| SEQ ID | oKM_0286 | tgagtacggtgggtttctggc | Sequencing: Forward primer |
| NO: 67 | | | 3 to sequence |
| | | | pKM133/pKM138 |
| SEQ ID | oKM_0287 | tcaggaatacgcagcacctgc | Sequencing: Forward primer |
| NO: 68 | | | 4 to sequence |
| | | | pKM133/pKM138 |
| SEQ ID | oKM_0292 | ccactgggccagcattgc | Sequencing: Forward primer |
| NO: 69 | | | 5 to sequence |
| | | | pKM133/pKM138 |
| SEQ ID | oKM_0293 | gtaccaactgttcgtgcgcg | Sequencing: Forward primer |
| NO: 70 | | | 6 to sequence |
| | | | pKM133/pKM138 |
| SEQ ID | oKM_0294 | gctgaccgtgcacatcagc | Sequencing: Forward primer |
| NO: 71 | | | 7 to sequence |
| | | | pKM133/pKM138 |
| SEQ ID | oKM_0295 | cgtgttactcgtcggatttccg | Sequencing: Forward primer |
| NO: 72 | | | 8 to sequence |
| | | | pKM133/pKM138 |
| SEQ ID | oKM_0321 | tctctagagtcgacctgcagaagcttGATGTGCTGGTTTGTGAC | Forward primer to insert PP- |
| NO: 73 | | GG | _0262 with fabH |
| | | | homologous arms into |
| | | | pKM26 at HindIII site |
| SEQ ID | oKM_0322 | gataacagggtaatagatctaagcttGACGCATCATCCAGACC | Reverse primer to insert PP- |
| NO: 74 | | GAT | _0262 with fabH |
| | | | homologous arms into |
| | | | pKM26 at HindIII site |
| SEQ ID | oKM_0323 | ctccaccgctgatgacatcag | Sequencing: Reverse primer |
| NO: 75 | | | for pTargetF |
| SEQ ID | oKM_0328 | tgaagacttggatgctagcaagcttctttctgctatggaggtcaggtatgatt | Forward primer to clone |
| NO: 76 | | | Plac::PP_0262 into pGW26 |
| | | | at HindIII/BamHI sites |
| SEQ ID | oKM_0329 | gggggcttttttattgcgcggatccttgtccagcagggttgtccac | Reverse primer to clone |
| NO: 77 | | | Plac::PP_0262 into pGW26 |
| | | | at HindIII/BamHI sites |
| SEQ ID | oKM_0332 | ATGCCTCAGCGGTGCTTAAA | Diagnostic: Forward primer |
| NO: 78 | | | to verify replacement of fabH |
| | | | with PP_0262 |
| SEQ ID | oKM_0333 | CGGAGAAACGACCTGACCTT | Diagnostic: Reverse primer |
| NO: 79 | | | to verify replacement of fabH |
| | | | with PP_0262 |
| SEQ ID | oLC_0005 | actggccgtcgttttacaacgt | Forward primer to linearize |
| NO: 80 | | | pK18sB |
| SEQ ID | oLC_0006 | gtcatagctgtttcctgtgtgaggcgt | Reverse primer to linearize |
| NO: 81 | | | pK18sB |
| SEQ ID | oLC_0157 | ctagGGTCTCaACTAGTATTATACCTAGGACTGAGCT | Reverse primer to linearize |
| NO: 82 | | AGCTGT | pTargetF backbone and |
| | | | introduce BsaI site |
| SEQ ID | oLC_0158 | ctagGGTCTCaGTTTTAGAGCTAGAAATAGCAAGTTA | Forward primer to linearize |
| NO: 83 | | AAATAAGGCTAGT | pTargetF backbone and |
| | | | introduce BsaI site |
| SEQ ID | oLC_0163 | TAGTCGGCGGAACGATTATTCTTG | CRISPR-Cas9 gRNA |
| NO: 84 | | | forward oligo for E. coli yiiD |
| SEQ ID | oLC_0164 | AAACcaagaataatcgttccgccg | CRISPR-Cas9 gRNA |
| NO: 85 | | | reverse oligo for E. coli yiiD |
| SEQ ID | oLC_0165 | ccgtggagggagaagtgacg | 5′ forward primer to |
| NO: 86 | | | generate ΔyiiD via CRISPR- |
| | | | Cas9 |
| SEQ ID | oLC_0166 | gctcatacctgcaaccagaatgt | 5′ reverse primer to |
| NO: 87 | | | generate ΔyiiD via CRISPR- |
| | | | Cas9 |
| SEQ ID | oLC_0167 | ttctggttgcaggtatgagctagctgatggagaggggcggt | 3′ forward primer to |
| NO: 88 | | | generate ΔyiiD via CRISPR- |
| | | | Cas9 |
| SEQ ID | oLC_0168 | ccacggtttaagagcaaacat | 3′ reverse primer to |
| NO: 89 | | | generate ΔyiiD via CRISPR- |
| | | | Cas9 |
| SEQ ID | oEUK114 | GCCGCGCGGCAGCCATATGTATACGAAGATTATTG | Forward cloning primer to |
| NO: 90 | | G | amplify P0A6R0 to produce |
| | | | pEUK069 |
| SEQ ID | oEUK115 | GTTAGCAGCCGGATCCCTAGAAACGAACCAGC | Reverse cloning primer to |
| NO: 91 | | | amplify P0A6R0 to produce |
| | | | pEUK069 |
| SEQ ID | oEUK116 | GCCGCGCGGCAGCCATATGACGCAATTTGCATTTG | Forward cloning primer to |
| NO: 92 | | | amplify P0AAI9 to produce |
| | | | pEUK070 |
| SEQ ID | oEUK117 | GTTAGCAGCCGGATCCTTAAAGCTCGAGCGCC | Reverse cloning primer to |
| NO: 93 | | | amplify P0AAI9 to produce |
| | | | pEUK070 |
| SEQ ID | 0EUK126 | GCCGCGCGGCAGCCATATGATTGGCATCAAAAGCA | Forward cloning primer to |
| NO: 94 | | | amplify PP_4379 to produce |
| | | | pEUK078 |
| SEQ ID | oEUK127 | GTTAGCAGCCGGATCCTCAGTCGCGGTACAGG | Reverse cloning primer to |
| NO: 95 | | | amplify PP_4379 to produce |
| | | | pEUK078 |
| SEQ ID | 0EUK128 | GCCGCGCGGCAGCCATATGCATAACGTCGTGATCA | Forward cloning primer to |
| NO: 96 | | | amplify PP_4379 to produce |
| | | | pEUK079 |
| SEQ ID | 0EUK129 | GTTAGCAGCCGGATCCTCAGCGCTTGCGC | Reverse cloning primer to |
| NO: 97 | | | amplify PP_4379 to produce |
| | | | pEUK079 |

TABLE 5
Details of plasmids disclosed herein.

| Plasmid | Utility | Plasmid Construction Details |
|---|---|---|
| pKM001 | Deletion of PP_4379 in P. | The 5′ targeting region was amplified with primer pair |
| | putida KT2440 | oKM0001/oKM0002, while the 3′ targeting region was amplified |
| | | with primer pair oKM0003/oKM0004; these fragments were |
| | | assembled into pK18sB amplified with primer pair |
| | | oLC0005/oLC0006. |
| pKM008 | Deletion of PP_0262 in P. | The 5′ targeting region was amplified with primer pair |
| | putida KT2440 | oKM0055/oKM0056, while the 3′ targeting region was amplified |
| | | with primer pair oKM0057/oKM0058; these fragments were |
| | | assembled into pK18sB amplified with primer pair |
| | | oLC0005/oLC0006. |
| pKM011 | Deletion of PP_4545 in P. | The 5′ targeting region was amplified with primer pair |
| | putida KT2440 | oKM0007/oKM0063, while the 3′ targeting region was amplified |
| | | with primer pair oKM0064/oKM0010; these fragments were |
| | | assembled into pK18sB amplified with primer pair |
| | | oLC0005/oLC0006. |
| pKM026 | Contains gRNA for targeting | pTargetF was linearized with oLC0157/oLC0158 and assembled |
| | E. coli fabH with CRISPR- | with annealed and phosphorylated oligos oKM0108/oKM0109 by |
| | Cas9 | Golden Gate reaction as described in Jiang, Y et al. 2015. |
| pKM037 | pBTL-2 derivative lacking | pBTL-2 was linearized using oKM0154/oKM0155 and the PCR |
| | BamHI site | product was self-assembled. |
| pKM038 | pBTL-2 driving expression of | pBTL-2 was linearized with oKM0180/oKM0181 and assembled |
| | PP_4379 | with PP_4379 amplified from P. putida KT2440 genomic DNA |
| | | with oKM0164/oKM0165. |
| pKM041 | pBTL-2 driving expression of | pBTL-2 was linearized with oKM0180/oKM0181 and assembled |
| | PP_4545 | with PP_4545 amplified from P. putida KT2440 genomic DNA |
| | | with oKM0166/oKM0167. |
| pKM044 | pBTL-2 driving expression of | pBTL-2 was linearized with oKM0180/oKM0181 and assembled |
| | E. coli fabH | with fabH amplified from E. coli MG1655 genomic DNA with |
| | | oKM0168/oKM0169. |
| pKM048 | pBTL-2 driving expression of | pBTL-2 was linearized with oKM0180/oKM0181 and assembled |
| | E. coli fabY | with fabY amplified from E. coli MG1655 genomic DNA with |
| | | oKM0170/oKM0171. |
| pKM062 | pKM037 derivative with | pKM037 was linearized using oKM0156/oKM0157 and the PCR |
| | insertion of BamHI site | product was self-assembled. |
| | downstream lac promoter | |
| pKM077- | These plasmids are | These plasmids are a subset of the P. putida KT2440 genomic |
| pKM096, | associated with rescued | library utilizing a pBTL-2 derivative expression vector, pKM062. |
| pKM109- | colony size of the E. coli | Detailed construction procedures are outlined in Methods. |
| pKM140 | MG1655 ΔfabH strain | |
| pKM097 | pBTL-2 driving expression of | pBTL-2 was linearized with oKM0180/oKM0181 and assembled |
| | PP_0262 | with PP_0262 amplified from P. putida KT2440 genomic DNA |
| | | with oKM0240/oKM0241. |
| pKM149 | pET-21b(+) overexpression of | pET-21b(+) was digested with NdeI and BamHI and assembled |
| | tagless PP_0262 | with PP_0262 amplified from P. putida KT2440 genomic DNA |
| | | with oKM0256/oKM0257. |
| pKM175 | pETDuet-1 overexpression of | pETDuet-1 was digested with BamHI and EcoRI and assembled |
| | E. coli 6xHis-AcpP | with acpP amplified from E. coli MG1655 genomic DNA with |
| | | oKM0278/oKM0279. |
| pKM181 | pETDuet-1 overexpression of | pKM175 was digested with NdeI and XhoI and assembled with |
| | E. coli 6xHis-AcpP and AcpS | acpS amplified from E. coli MG1655 genomic DNA with |
| | | oKM0280/oKM0281. |
| pKM191 | pBTL-2 driving expression of | Synthesized by Twist Bioscience |
| | PP_0262 (S2A) as in pKM097 | |
| pKM192 | pBTL-2 driving expression of | Synthesized by Twist Bioscience |
| | PP_0262 (S10A) as in | |
| | pKM097 | |
| pKM193 | pBTL-2 driving expression of | Synthesized by Twist Bioscience |
| | PP_0262 (L18A) as in | |
| | pKM097 | |
| pKM194 | pBTL-2 driving expression of | Synthesized by Twist Bioscience |
| | PP_0262 (V26A) as in | |
| | pKM097 | |
| pKM195 | pBTL-2 driving expression of | Synthesized by Twist Bioscience |
| | PP_0262 (L34A) as in | |
| | pKM097 | |
| pKM196 | pBTL-2 driving expression of | Synthesized by Twist Bioscience |
| | PP_0262 (V44A) as in | |
| | pKM097 | |
| pKM197 | pBTL-2 driving expression of | Synthesized by Twist Bioscience |
| | PP_0262 (G52A) as in | |
| | pKM097 | |
| pKM198 | pBTL-2 driving expression of | Synthesized by Twist Bioscience |
| | PP_0262 (V62A) as in | |
| | pKM097 | |
| pKM199 | pBTL-2 driving expression of | Synthesized by Twist Bioscience |
| | PP_0262 (L71A) as in | |
| | pKM097 | |
| pKM200 | pBTL-2 driving expression of | Synthesized by Twist Bioscience |
| | PP_0262 (G79A) as in | |
| | pKM097 | |
| pKM201 | pBTL-2 driving expression of | Synthesized by Twist Bioscience |
| | PP_0262 (Q87A) as in | |
| | pKM097 | |
| pKM202 | pBTL-2 driving expression of | Synthesized by Twist Bioscience |
| | PP_0262 (T95A) as in | |
| | pKM097 | |
| pKM203 | pBTL-2 driving expression of | Synthesized by Twist Bioscience |
| | PP_0262 (T3A) as in pKM097 | |
| pKM204 | pBTL-2 driving expression of | Synthesized by Twist Bioscience |
| | PP_0262 (V11A) as in | |
| | pKM097 | |
| pKM205 | pBTL-2 driving expression of | Synthesized by Twist Bioscience |
| | PP_0262 (T19A) as in | |
| | pKM097 | |
| pKM206 | pBTL-2 driving expression of | Synthesized by Twist Bioscience |
| | PP_0262 (I27A) as in pKM097 | |
| pKM207 | pBTL-2 driving expression of | Synthesized by Twist Bioscience |
| | PP_0262 (R35A) as in | |
| | pKM097 | |
| pKM208 | pBTL-2 driving expression of | Synthesized by Twist Bioscience |
| | PP_0262 (N45A) as in | |
| | pKM097 | |
| pKM209 | pBTL-2 driving expression of | Synthesized by Twist Bioscience |
| | PP_0262 (G53A) as in | |
| | pKM097 | |
| pKM210 | pBTL-2 driving expression of | Synthesized by Twist Bioscience |
| | PP_0262 (G63A) as in | |
| | pKM097 | |
| pKM211 | pBTL-2 driving expression of | Synthesized by Twist Bioscience |
| | PP_0262 (R72A) as in | |
| | pKM097 | |
| pKM212 | pBTL-2 driving expression of | Synthesized by Twist Bioscience |
| | PP_0262 (H80A) as in | |
| | pKM097 | |
| pKM213 | pBTL-2 driving expression of | Synthesized by Twist Bioscience |
| | PP_0262 (I88A) as in pKM097 | |
| pKM214 | pBTL-2 driving expression of | Synthesized by Twist Bioscience |
| | PP_0262 (G96A) as in | |
| | pKM097 | |
| pKM215 | pBTL-2 driving expression of | Synthesized by Twist Bioscience |
| | PP_0262 (D4A) as in pKM097 | |
| pKM216 | pBTL-2 driving expression of | Synthesized by Twist Bioscience |
| | PP_0262 (L12A) as in | |
| | pKM097 | |
| pKM217 | pBTL-2 driving expression of | Synthesized by Twist Bioscience |
| | PP_0262 (R20A) as in | |
| | pKM097 | |
| pKM218 | pBTL-2 driving expression of | Synthesized by Twist Bioscience |
| | PP_0262 (D28A) as in | |
| | pKM097 | |
| pKM219 | pBTL-2 driving expression of | Synthesized by Twist Bioscience |
| | PP_0262 (L36A) as in | |
| | pKM097 | |
| pKM220 | pBTL-2 driving expression of | Synthesized by Twist Bioscience |
| | PP_0262 (H46A) as in | |
| | pKM097 | |
| pKM221 | pBTL-2 driving expression of | Synthesized by Twist Bioscience |
| | PP_0262 (S54A) as in | |
| | pKM097 | |
| pKM222 | pBTL-2 driving expression of | Synthesized by Twist Bioscience |
| | PP_0262 (W64A) as in | |
| | pKM097 | |
| pKM223 | pBTL-2 driving expression of | Synthesized by Twist Bioscience |
| | PP_0262 (E73A) as in | |
| | pKM097 | |
| pKM224 | pBTL-2 driving expression of | Synthesized by Twist Bioscience |
| | PP_0262 (I81A) as in pKM097 | |
| pKM225 | pBTL-2 driving expression of | Synthesized by Twist Bioscience |
| | PP_0262 (S89A) as in | |
| | pKM097 | |
| pKM226 | pBTL-2 driving expression of | Synthesized by Twist Bioscience |
| | PP_0262 (T97A) as in | |
| | pKM097 | |
| pKM227 | pBTL-2 driving expression of | Synthesized by Twist Bioscience |
| | PP_0262 (S5A) as in pKM097 | |
| pKM228 | pBTL-2 driving expression of | Synthesized by Twist Bioscience |
| | PP_0262 (H13A) as in | |
| | pKM097 | |
| pKM229 | pBTL-2 driving expression of | Synthesized by Twist Bioscience |
| | PP_0262 (E21A) as in | |
| | pKM097 | |
| pKM230 | pBTL-2 driving expression of | Synthesized by Twist Bioscience |
| | PP_0262 (W29A) as in | |
| | pKM097 | |
| pKM231 | pBTL-2 driving expression of | Synthesized by Twist Bioscience |
| | PP_0262 (Q37A) as in | |
| | pKM097 | |
| pKM232 | pBTL-2 driving expression of | Synthesized by Twist Bioscience |
| | PP_0262 (K47A) as in | |
| | pKM097 | |
| pKM233 | pBTL-2 driving expression of | Synthesized by Twist Bioscience |
| | PP_0262 (L55A) as in | |
| | pKM097 | |
| pKM234 | pBTL-2 driving expression of | Synthesized by Twist Bioscience |
| | PP_0262 (G65A) as in | |
| | pKM097 | |
| pKM235 | pBTL-2 driving expression of | Synthesized by Twist Bioscience |
| | PP_0262 (L74A) as in | |
| | pKM097 | |
| pKM236 | pBTL-2 driving expression of | Synthesized by Twist Bioscience |
| | PP_0262 (V82A) as in | |
| | pKM097 | |
| pKM237 | pBTL-2 driving expression of | Synthesized by Twist Bioscience |
| | PP_0262 (Y90A) as in | |
| | pKM097 | |
| pKM238 | pBTL-2 driving expression of | Synthesized by Twist Bioscience |
| | PP_0262 (V99A) as in | |
| | pKM097 | |
| pKM239 | pBTL-2 driving expression of | Synthesized by Twist Bioscience |
| | PP_0262 (Q6A) as in pKM097 | |
| pKM240 | pBTL-2 driving expression of | Synthesized by Twist Bioscience |
| | PP_0262 (S14A) as in | |
| | pKM097 | |
| pKM241 | pBTL-2 driving expression of | Synthesized by Twist Bioscience |
| | PP_0262 (M22A) as in | |
| | pKM097 | |
| pKM242 | pBTL-2 driving expression of | Synthesized by Twist Bioscience |
| | PP_0262 (Q30A) as in | |
| | pKM097 | |
| pKM243 | pBTL-2 driving expression of | Synthesized by Twist Bioscience |
| | PP_0262 (L38A) as in | |
| | pKM097 | |
| pKM244 | pBTL-2 driving expression of | Synthesized by Twist Bioscience |
| | PP_0262 (S48A) as in | |
| | pKM097 | |
| pKM245 | pBTL-2 driving expression of | Synthesized by Twist Bioscience |
| | PP_0262 (Y56A) as in | |
| | pKM097 | |
| pKM246 | pBTL-2 driving expression of | Synthesized by Twist Bioscience |
| | PP_0262 (W66A) as in | |
| | pKM097 | |
| pKM247 | pBTL-2 driving expression of | Synthesized by Twist Bioscience |
| | PP_0262 (G75A) as in | |
| | pKM097 | |
| pKM248 | pBTL-2 driving expression of | Synthesized by Twist Bioscience |
| | PP_0262 (I83A) as in pKM097 | |
| pKM249 | pBTL-2 driving expression of | Synthesized by Twist Bioscience |
| | PP_0262 (P91A) as in | |
| | pKM097 | |
| pKM250 | pBTL-2 driving expression of | Synthesized by Twist Bioscience |
| | PP_0262 (R101A) as in | |
| | pKM097 | |
| pKM251 | pBTL-2 driving expression of | Synthesized by Twist Bioscience |
| | PP_0262 (Y7A) as in pKM097 | |
| pKM252 | pBTL-2 driving expression of | Synthesized by Twist Bioscience |
| | PP_0262 (D15A) as in | |
| | pKM097 | |
| pKM253 | pBTL-2 driving expression of | Synthesized by Twist Bioscience |
| | PP_0262 (G23A) as in | |
| | pKM097 | |
| pKM254 | pBTL-2 driving expression of | Synthesized by Twist Bioscience |
| | PP_0262 (Q31A) as in | |
| | pKM097 | |
| pKM255 | pBTL-2 driving expression of | Synthesized by Twist Bioscience |
| | PP_0262 (P39A) as in | |
| | pKM097 | |
| pKM256 | pBTL-2 driving expression of | Synthesized by Twist Bioscience |
| | PP_0262 (T49A) as in | |
| | pKM097 | |
| pKM257 | pBTL-2 driving expression of | Synthesized by Twist Bioscience |
| | PP_0262 (C57A) as in | |
| | pKM097 | |
| pKM258 | pBTL-2 driving expression of | Synthesized by Twist Bioscience |
| | PP_0262 (L67A) as in | |
| | pKM097 | |
| pKM259 | pBTL-2 driving expression of | Synthesized by Twist Bioscience |
| | PP_0262 (I76A) as in pKM097 | |
| pKM260 | pBTL-2 driving expression of | Synthesized by Twist Bioscience |
| | PP_0262 (Q84A) as in | |
| | pKM097 | |
| pKM261 | pBTL-2 driving expression of | Synthesized by Twist Bioscience |
| | PP_0262 (L92A) as in | |
| | pKM097 | |
| pKM262 | pBTL-2 driving expression of | Synthesized by Twist Bioscience |
| | PP_0262 (C102A) as in | |
| | pKM097 | |
| pKM263 | pBTL-2 driving expression of | Synthesized by Twist Bioscience |
| | PP_0262 (L8A) as in pKM097 | |
| pKM264 | pBTL-2 driving expression of | Synthesized by Twist Bioscience |
| | PP_0262 (I16A) as in pKM097 | |
| pKM265 | pBTL-2 driving expression of | Synthesized by Twist Bioscience |
| | PP_0262 (L24A) as in | |
| | pKM097 | |
| pKM266 | pBTL-2 driving expression of | Synthesized by Twist Bioscience |
| | PP_0262 (H32A) as in | |
| | pKM097 | |
| pKM267 | pBTL-2 driving expression of | Synthesized by Twist Bioscience |
| | PP_0262 (L40A) as in | |
| | pKM097 | |
| pKM268 | pBTL-2 driving expression of | Synthesized by Twist Bioscience |
| | PP_0262 (M50A) as in | |
| | pKM097 | |
| pKM269 | pBTL-2 driving expression of | Synthesized by Twist Bioscience |
| | PP_0262 (V60A) as in | |
| | pKM097 | |
| pKM270 | pBTL-2 driving expression of | Synthesized by Twist Bioscience |
| | PP_0262 (H68A) as in | |
| | pKM097 | |
| pKM271 | pBTL-2 driving expression of | Synthesized by Twist Bioscience |
| | PP_0262 (D77A) as in | |
| | pKM097 | |
| pKM272 | pBTL-2 driving expression of | Synthesized by Twist Bioscience |
| | PP_0262 (E85A) as in | |
| | pKM097 | |
| pKM273 | pBTL-2 driving expression of | Synthesized by Twist Bioscience |
| | PP_0262 (P93A) as in | |
| | pKM097 | |
| pKM274 | pBTL-2 driving expression of | Synthesized by Twist Bioscience |
| | PP_0262 (P105A) as in | |
| | pKM097 | |
| pKM275 | pBTL-2 driving expression of | Synthesized by Twist Bioscience |
| | PP_0262 (Q9A) as in pKM097 | |
| pKM276 | pBTL-2 driving expression of | Synthesized by Twist Bioscience |
| | PP_0262 (P17A) as in | |
| | pKM097 | |
| pKM277 | pBTL-2 driving expression of | Synthesized by Twist Bioscience |
| | PP_0262 (E25A) as in | |
| | pKM097 | |
| pKM278 | pBTL-2 driving expression of | Synthesized by Twist Bioscience |
| | PP_0262 (T33A) as in | |
| | pKM097 | |
| pKM279 | pBTL-2 driving expression of | Synthesized by Twist Bioscience |
| | PP_0262 (N43A) as in | |
| | pKM097 | |
| pKM280 | pBTL-2 driving expression of | Synthesized by Twist Bioscience |
| | PP_0262 (F51A) as in | |
| | pKM097 | |
| pKM281 | pBTL-2 driving expression of | Synthesized by Twist Bioscience |
| | PP_0262 (L61A) as in | |
| | pKM097 | |
| pKM282 | pBTL-2 driving expression of | Synthesized by Twist Bioscience |
| | PP_0262 (R70A) as in | |
| | pKM097 | |
| pKM283 | pBTL-2 driving expression of | Synthesized by Twist Bioscience |
| | PP_0262 (D78A) as in | |
| | pKM097 | |
| pKM284 | pBTL-2 driving expression of | Synthesized by Twist Bioscience |
| | PP_0262 (G86A) as in | |
| | pKM097 | |
| pKM285 | pBTL-2 driving expression of | Synthesized by Twist Bioscience |
| | PP_0262 (V94A) as in | |
| | pKM097 | |
| pKM286 | pBTL-2 driving expression of | Synthesized by Twist Bioscience |
| | PP_0262 (D106A) as in | |
| | pKM097 | |
| pKM287 | pBTL-2 driving expression of | Synthesized by Twist Bioscience |
| | PP_0262 (E107A) as in | |
| | pKM097 | |
| pKM288 | pBTL-2 driving expression of | Synthesized by Twist Bioscience |
| | PP_0262 (T115A) as in | |
| | pKM097 | |
| pKM289 | pBTL-2 driving expression of | Synthesized by Twist Bioscience |
| | PP_0262 (R124A) as in | |
| | pKM097 | |
| pKM290 | pBTL-2 driving expression of | Synthesized by Twist Bioscience |
| | PP_0262 (N133A) as in | |
| | pKM097 | |
| pKM291 | pBTL-2 driving expression of | Synthesized by Twist Bioscience |
| | PP_0262 (R142A) as in | |
| | pKM097 | |
| pKM292 | pBTL-2 driving expression of | Synthesized by Twist Bioscience |
| | PP_0262 (H150A) as in | |
| | pKM097 | |
| pKM293 | pBTL-2 driving expression of | Synthesized by Twist Bioscience |
| | PP_0262 (K108A) as in | |
| | pKM097 | |
| pKM294 | pBTL-2 driving expression of | Synthesized by Twist Bioscience |
| | PP_0262 (L116A) as in | |
| | pKM097 | |
| pKM295 | pBTL-2 driving expression of | Synthesized by Twist Bioscience |
| | PP_0262 (L125A) as in | |
| | pKM097 | |
| pKM296 | pBTL-2 driving expression of | Synthesized by Twist Bioscience |
| | PP_0262 (D134A) as in | |
| | pKM097 | |
| pKM297 | pBTL-2 driving expression of | Synthesized by Twist Bioscience |
| | PP_0262 (F143A) as in | |
| | pKM097 | |
| pKM298 | pBTL-2 driving expression of | Synthesized by Twist Bioscience |
| | PP_0262 (R151A) as in | |
| | pKM097 | |
| pKM299 | pBTL-2 driving expression of | Synthesized by Twist Bioscience |
| | PP_0262 (T109A) as in | |
| | pKM097 | |
| pKM300 | pBTL-2 driving expression of | Synthesized by Twist Bioscience |
| | PP_0262 (Y117A) as in | |
| | pKM097 | |
| pKM301 | pBTL-2 driving expression of | Synthesized by Twist Bioscience |
| | PP_0262 (T126A) as in | |
| | pKM097 | |
| pKM302 | pBTL-2 driving expression of | Synthesized by Twist Bioscience |
| | PP_0262 (G135A) as in | |
| | pKM097 | |
| pKM303 | pBTL-2 driving expression of | Synthesized by Twist Bioscience |
| | PP_0262 (S144A) as in | |
| | pKM097 | |
| pKM304 | pBTL-2 driving expression of | Synthesized by Twist Bioscience |
| | PP_0262 (W110A) as in | |
| | pKM097 | |
| pKM305 | pBTL-2 driving expression of | Synthesized by Twist Bioscience |
| | PP_0262 (Q118A) as in | |
| | pKM097 | |
| pKM306 | pBTL-2 driving expression of | Synthesized by Twist Bioscience |
| | PP_0262 (E128A) as in | |
| | pKM097 | |
| pKM307 | pBTL-2 driving expression of | Synthesized by Twist Bioscience |
| | PP_0262 (S136A) as in | |
| | pKM097 | |
| pKM308 | pBTL-2 driving expression of | Synthesized by Twist Bioscience |
| | PP_0262 (G145A) as in | |
| | pKM097 | |
| pKM309 | pBTL-2 driving expression of | Synthesized by Twist Bioscience |
| | PP_0262 (E111A) as in | |
| | pKM097 | |
| pKM310 | pBTL-2 driving expression of | Synthesized by Twist Bioscience |
| | PP_0262 (R119A) as in | |
| | pKM097 | |
| pKM311 | pBTL-2 driving expression of | Synthesized by Twist Bioscience |
| | PP_0262 (T129A) as in | |
| | pKM097 | |
| pKM312 | pBTL-2 driving expression of | Synthesized by Twist Bioscience |
| | PP_0262 (D137A) as in | |
| | pKM097 | |
| pKM313 | pBTL-2 driving expression of | Synthesized by Twist Bioscience |
| | PP_0262 (Q146A) as in | |
| | pKM097 | |
| pKM314 | pBTL-2 driving expression of | Synthesized by Twist Bioscience |
| | PP_0262 (R112A) as in | |
| | pKM097 | |
| pKM315 | pBTL-2 driving expression of | Synthesized by Twist Bioscience |
| | PP_0262 (R120A) as in | |
| | pKM097 | |
| pKM316 | pBTL-2 driving expression of | Synthesized by Twist Bioscience |
| | PP_0262 (T130A) as in | |
| | pKM097 | |
| pKM317 | pBTL-2 driving expression of | Synthesized by Twist Bioscience |
| | PP_0262 (E138A) as in | |
| | pKM097 | |
| pKM318 | pBTL-2 driving expression of | Synthesized by Twist Bioscience |
| | PP_0262 (Y147A) as in | |
| | pKM097 | |
| pKM319 | pBTL-2 driving expression of | Synthesized by Twist Bioscience |
| | PP_0262 (F113A) as in | |
| | pKM097 | |
| pKM320 | pBTL-2 driving expression of | Synthesized by Twist Bioscience |
| | PP_0262 (G121A) as in | |
| | pKM097 | |
| pKM321 | pBTL-2 driving expression of | Synthesized by Twist Bioscience |
| | PP_0262 (V131A) as in | |
| | pKM097 | |
| pKM322 | pBTL-2 driving expression of | Synthesized by Twist Bioscience |
| | PP_0262 (P139A) as in | |
| | pKM097 | |
| pKM323 | pBTL-2 driving expression of | Synthesized by Twist Bioscience |
| | PP_0262 (V148A) as in | |
| | pKM097 | |
| pKM324 | pBTL-2 driving expression of | Synthesized by Twist Bioscience |
| | PP_0262 (L114A) as in | |
| | pKM097 | |
| pKM325 | pBTL-2 driving expression of | Synthesized by Twist Bioscience |
| | PP_0262 (R122A) as in | |
| | pKM097 | |
| pKM326 | pBTL-2 driving expression of | Synthesized by Twist Bioscience |
| | PP_0262 (S132A) as in | |
| | pKM097 | |
| pKM327 | pBTL-2 driving expression of | Synthesized by Twist Bioscience |
| | PP_0262 (V141A) as in | |
| | pKM097 | |
| pKM328 | pBTL-2 driving expression of | Synthesized by Twist Bioscience |
| | PP_0262 (L149A) as in | |
| | pKM097 | |
| pKM329 | pBTL-2 driving expression of | Synthesized by Twist Bioscience |
| | PP_0262 (L127A) as in | |
| | pKM097 | |
| pKM330 | pBTL-2 driving expression of | Synthesized by Twist Bioscience |
| | PP_0262 (L69A) as in | |
| | pKM097 | |
| pKM338 | Contains gRNA for targeting | pKM026 was linearized with HindIII. PP_0262 was amplified |
| | E. coli fabH and template for | from P. putida KT2440 genomic DNA with oKM0265/oKM0266. |
| | replacement with PP_0262 via | The 5′ and 3′ flanks of fabH were amplified from E. coli MG1655 |
| | CRISPR-Cas9 | genomic DNA with oKM0321/oKM0259 and oKM0260/oKM0322, |
| | | respectively. All four DNA fragments were then assembled. |
| pKM341 | Plasmid expressing PP_0262 | pGW26 (Elmore, J.R. et al. 2020) was digested with HindIII and |
| | that has temperature sensitive | BamHI. Plac::PP_0262 was amplified from pKM097 using |
| | replication in P. putida KT2440 | oKM0328/oKM0329 and assembled with digested pGW26. |
| pKM354 | pBTL-2 driving expression of | Synthesized by Twist Bioscience |
| | PP_0262 (L8I) as in pKM097 | |
| pKM355 | pBTL-2 driving expression of | Synthesized by Twist Bioscience |
| | PP_0262 (L8V) as in pKM097 | |
| pKM356 | pBTL-2 driving expression of | Synthesized by Twist Bioscience |
| | PP_0262 (H13K) as in | |
| | pKM097 | |
| pKM357 | pBTL-2 driving expression of | Synthesized by Twist Bioscience |
| | PP_0262 (H13R) as in | |
| | pKM097 | |
| pKM358 | pBTL-2 driving expression of | Synthesized by Twist Bioscience |
| | PP_0262 (I16L) as in pKM097 | |
| pKM359 | pBTL-2 driving expression of | Synthesized by Twist Bioscience |
| | PP_0262 (I16V) as in pKM097 | |
| pKM360 | pBTL-2 driving expression of | Synthesized by Twist Bioscience |
| | PP_0262 (L18I) as in pKM097 | |
| pKM361 | pBTL-2 driving expression of | Synthesized by Twist Bioscience |
| | PP_0262 (L18V) as in | |
| | pKM097 | |
| pKM362 | pBTL-2 driving expression of | Synthesized by Twist Bioscience |
| | PP_0262 (N43Q) as in | |
| | pKM097 | |
| pKM363 | pBTL-2 driving expression of | Synthesized by Twist Bioscience |
| | PP_0262 (N43D) as in | |
| | pKM097 | |
| pKM364 | pBTL-2 driving expression of | Synthesized by Twist Bioscience |
| | PP_0262 (N45Q) as in | |
| | pKM097 | |
| pKM365 | pBTL-2 driving expression of | Synthesized by Twist Bioscience |
| | PP_0262 (N45D) as in | |
| | pKM097 | |
| pKM366 | pBTL-2 driving expression of | Synthesized by Twist Bioscience |
| | PP_0262 (W64F) as in | |
| | pKM097 | |
| pKM367 | pBTL-2 driving expression of | Synthesized by Twist Bioscience |
| | PP_0262 (W64Y) as in | |
| | pKM097 | |
| pKM368 | pBTL-2 driving expression of | Synthesized by Twist Bioscience |
| | PP_0262 (Y90F) as in | |
| | pKM097 | |
| pKM369 | pBTL-2 driving expression of | Synthesized by Twist Bioscience |
| | PP_0262 (Y90W) as in | |
| | pKM097 | |
| pKM370 | pBTL-2 driving expression of | Synthesized by Twist Bioscience |
| | PP_0262 (R124K) as in | |
| | pKM097 | |
| pKM371 | pBTL-2 driving expression of | Synthesized by Twist Bioscience |
| | PP_0262 (R124H) as in | |
| | pKM097 | |
| pKM372 | pBTL-2 driving expression of | Synthesized by Twist Bioscience |
| | PP_0262 (Y147F) as in | |
| | pKM097 | |
| pKM373 | pBTL-2 driving expression of | Synthesized by Twist Bioscience |
| | PP_0262 (Y147W) as in | |
| | pKM097 | |
| pKM376 | pET-21b(+) overexpression of | pET-21b(+) was digested with NdeI and BamHI and assembled |
| | tagless N45A PP_0262 | with N45A PP_0262 amplified from pKM208 with |
| | | oKM0256/oKM0257. |
| pKM413 | pBTL-2 driving expression of | Synthesized by Twist Bioscience |
| | PP_0262 homolog from | |
| | Acaryochloris marina | |
| pKM414 | pBTL-2 driving expression of | Synthesized by Twist Bioscience |
| | PP_0262 homolog from | |
| | Arcobacter butzleri | |
| pKM415 | pBTL-2 driving expression of | Synthesized by Twist Bioscience |
| | PP_0262 homolog from | |
| | Arthrobacter stackebrandtii | |
| pKM416 | pBTL-2 driving expression of | Synthesized by Twist Bioscience |
| | PP_0262 homolog from | |
| | Corallococcus coralloides | |
| pKM417 | pBTL-2 driving expression of | Synthesized by Twist Bioscience |
| | PP_0262 homolog from | |
| | Parachlamydia | |
| | acanthamoebae | |
| pKM418 | pBTL-2 driving expression of | Synthesized by Twist Bioscience |
| | PP_0262 homolog from | |
| | Bdellovibrio bacteriovorus | |
| pKM419 | pBTL-2 driving expression of | Synthesized by Twist Bioscience |
| | PP_0262 homolog from | |
| | Marispirochaeta aestuarii | |
| pKM420 | pBTL-2 driving expression of | Synthesized by Twist Bioscience |
| | PP_0262 homolog from | |
| | Klebsiella pneumoniae | |
| pKM421 | pBTL-2 driving expression of | Synthesized by Twist Bioscience |
| | PP_0262 homolog from | |
| | Chlorobaculum tepidum | |
| pKM422 | pBTL-2 driving expression of | Synthesized by Twist Bioscience |
| | PP_0262 homolog from | |
| | Clostridium beijerinckii | |
| pKM423 | pBTL-2 driving expression of | Synthesized by Twist Bioscience |
| | PP_0262 homolog from | |
| | Prosthecobacter fusiformis | |
| pKM424 | pBTL-2 driving expression of | Synthesized by Twist Bioscience |
| | PP_0262 homolog from | |
| | Fimbriimonas ginsengisoli | |
| pKM425 | pBTL-2 driving expression of | Synthesized by Twist Bioscience |
| | PP_0262 homolog from | |
| | Rickettsiales endosymbiont of | |
| | Stachyamoeba lipophora | |
| pKM426 | pBTL-2 driving expression of | Synthesized by Twist Bioscience |
| | PP_0262 homolog from | |
| | Ktedonobacter racemifer | |
| pKM427 | pBTL-2 driving expression of | Synthesized by Twist Bioscience |
| | PP_0262 homolog from | |
| | Salmonella enterica | |
| pKM428 | pBTL-2 driving expression of | Synthesized by Twist Bioscience |
| | PP_0262 homolog from | |
| | Neisseria sicca | |
| pKM429 | pBTL-2 driving expression of | Synthesized by Twist Bioscience |
| | PP_0262 homolog from | |
| | Mariprofundus ferrooxydans | |
| pKM430 | pBTL-2 driving expression of | Synthesized by Twist Bioscience |
| | PP_0262 homolog from | |
| | Aeromonas hydrophila | |
| pKM431 | pBTL-2 driving expression of | Synthesized by Twist Bioscience |
| | PP_0262 homolog from | |
| | Pseudomonas syringae | |
| pKM432 | pBTL-2 driving expression of | Synthesized by Twist Bioscience |
| | PP_0262 homolog from | |
| | Sebaldella termitidis | |
| pKM433 | pBTL-2 driving expression of | Synthesized by Twist Bioscience |
| | PP_0262 homolog from Vibrio | |
| | parahaemolyticus | |
| pKM434 | pBTL-2 driving expression of | Synthesized by Twist Bioscience |
| | PP_0262 homolog from | |
| | Salinibacter ruber | |
| pEUK069 | Overexpression of EcFabH | pET15b (+) T7 expression vector containing P0A6R0 amplified |
| | | from MG1655 gDNA (oEUK114 & oEUK115) using Gibson |
| | | assembly between insertion points: NdeI and BamHI; Gene |
| | | product: His-thrombin^{pro}-EcFabH |
| pEUK070 | Overexpression of EcFabD | pET15b (+) T7 expression vector containing P0AAI9 amplified |
| | | from MG1655 gDNA (oEUK116 & oEUK117) using Gibson |
| | | assembly between inserion points: NdeI and BamHI; Gene |
| | | product: His-thrombin^{pro}-EcFabD |
| pEUK078 | Heterologous expression of | pET15b (+) T7 expression vector containing PP_4379 amplified |
| | PP_4379 in E. coli | from KT2440 gDNA (oEUK126 & oEUK127) using Gibson |
| | | assembly between insertion points: NdeI and BamHI; Gene |
| | | product: His-thrombin^{pro}-FabH1 |
| pEUK079 | Heterologous expression of | pET15 (+) T7 expression vector containing PP_4545 amplified |
| | PP_4545 in E. coli | from KT2440 gDNA (oEUK128 & oEUK129) using Gibson |
| | | assembly between insertion points: NdeI and BamHI; Gene |
| | | product: His-thrombin^{pro}-FabH2 |
| pEUK092 | pET-21b(+) overexpression of | pET21b (+) T7 expression vector containing H46A PP_0262 |
| | tagless H46A PP_0262 | amplified from pKM220 (oKM_0256 & oKM_0257) using Gibson |
| | | assembly between insertion points: NdeI and BamHI. |
| pET-Sfp | Heterologous expression of | T7 expression vector containing Sfp procured from Addgene |
| | Sfp in E. coli | #159617. |
| pLC027 | Contains gRNA for targeting | pTargetF was linearized with oLC0157/oLC0158 and assembled |
| | E. coli yiiD with CRISPR-Cas9 | with annealed and phosphorylated oligos oKM0108/oKM0109 by |
| | | Golden Gate reaction as described in Jiang, Y et al. 2015. |

Strain construction. CRISPR-Cas9-mediated deletions and replacements in E. coli MG1655 were carried out as previously described. P. putida KT2440 genomic deletions were carried out using a selection (nptII, kanamycin), counterselection (sacB, sucrose) approach outlined previously, utilizing plasmid pK18sB. Genomic modifications were confirmed by colony PCR using MyTaq™ HS Red Mix (Bioline). Proper counterselection in P. putida was further verified by ensuring strains were kanamycin-sensitive. Strains used in this study are described in Table 6.

TABLE 6
Additional details of strains disclosed herein.

| Strain | Genotype | Strain Construction Details |
|---|---|---|
| KM128 | E. coli MG1655 | E. coli MG1655 was transformed with pCas (Jiang, Y et al. |
| | ΔfabH | 2015). The resulting strain was co-transformed with pKM026 |
| | | and DNA template generated with primers oKM_0110, |
| | | oKM_0175, oKM_0176, and oKM_0113. Deletion of fabH was |
| | | verified using oKM_0110 and oKM_0113. pKM026 and pCas |
| | | were cured using IPTG and 37° C. incubation, respectively. |
| KM417 | P. putida KT2440 | ATCC 47054 was propagated in liquid LB medium, generating |
| | wild type | KM385. A single colony isolate of streaked KM385 was |
| | | propagated in liquid LB medium, generating KM417. |
| KM429 | P. putida KT2440 | PP_0262 was deleted from KM417 with pKM008. This deletion |
| | ΔPP_0262 | was confirmed by diagnostic colony PCR with primer pair |
| | | oKM_0059/oKM_0060. |
| KM433 | P. putida KT2440 | PP_4545 was deleted from KM417 with pKM011. This deletion |
| | ΔPP_4545 | was confirmed by diagnostic colony PCR with primer pair |
| | | oKM_0011/oKM_0012. |
| KM436 | P. putida KT2440 | PP_4379 was deleted from KM417 with pKM001. This deletion |
| | ΔPP_4379 | was confirmed by diagnostic colony PCR with primer pair |
| | | oKM_0005/oKM_0006. |
| KM452 | E. coli MG1655 | E. coli MG1655 was transformed with pCas (Jiang, Y et al. |
| | ΔyiiD | 2015). The resulting strain was co-transformed with pLC027 and |
| | | DNA template generated with primers oLC_0165-oLC_0168. |
| | | Deletion of fabY was verified using oLC_0165 and oLC_0168. |
| | | pLC027 and pCas were cured using IPTG and 37° C. incubation, |
| | | respectively. |
| KM454 | P. putida KT2440 | PP_4545 was deleted from KM429 with pKM011. This deletion |
| | ΔPP_0262 ΔPP_4545 | was confirmed by diagnostic colony PCR with primer pair |
| | | oKM_0011/oKM_0012. |
| KM460 | P. putida KT2440 | PP_4545 was deleted from KM436 with pKM011. This deletion |
| | ΔPP_4379 ΔPP_4545 | was confirmed by diagnostic colony PCR with primer pair |
| | | oKM_0011/oKM_0012. |
| KM461 | E. coli MG1655 | KM452 was transformed with pKM338. Replacement of fabH |
| | ΔyiiD ΔfabH:PP_0262 | with PP_0262 was verified using oKM_0110 and oKM_0113. |
| | | pKM338 and pCas were cured using IPTG and 37° C. incubation, |
| | | respectively. |
| KM493 | P. putida KT2440 | PP_4379 was deleted from KM429 with pKM001. This deletion |
| | ΔPP_0262 ΔPP_4379 | was confirmed by diagnostic colony PCR with primer pair |
| | | oKM_0005/oKM_0006. |
| KM555 | P. putida KT2440 | pKM341 was transformed into KM460. PP_0262 was |
| | ΔPP_4379 ΔPP_4545 | subsequently deleted with pKM008. This deletion was confirmed |
| | ΔPP_0262 + | by diagnostic colony PCR with primer pair oKM_0059/ |
| | pKM341 (PP_0262^{ts}) | oKM_0060. |

Microplate reader experiments. Microplate reader experiments were performed by inoculating seed cultures from glycerol stocks into 10 mL of LB Broth (Miller; Sigma-Aldrich) with appropriate antibiotics in a 125 mL flask and incubating overnight at 30° C. (P. putida) or 37° C. (E. coli) and 225 rpm. Overnight cultures were then used to inoculate a second seed culture into 10 mL of LB Broth (Miller; Sigma-Aldrich) with appropriate antibiotics in a 125 mL flask starting at an optical density of 0.2 measured at 600 nm (0D600) using a GENESYS™ 140 Visible Light Spectrophotometer (Thermo Scientific). The second seed cultures were incubated at 30° C. (P. putida) or 37° C. (E. coli) at 225 rpm for 2-4 hrs until an OD600 of approximately 2 was reached. The second seed cultures were centrifuged at 8,000 rpm for 3 min, the pellets were washed with 1× M9, minimal salts (Sigma-Aldrich) three times, and resuspended to an OD600=3.0. Cells (10 μL) were transferred to five replicate wells in a 100-well Honeycomb 2 plate (Growth Curves USA) containing 290 μL of growth medium, corresponding to initial OD600=0.1. E. coli strains were grown in LB Broth (Miller; Sigma-Aldrich) at 37° C. and supplemented with 50 μg/mL kanamycin and 0.1 mM isopropylthio-β-galactoside (IPTG; GoldBio) when appropriate. P. putida strains were grown in modified M9 minimal media containing 6.78 g/L Na2HPO4, 3 g/L KH2PO4, 1 g/L NH4C1, 0.5 g/L NaCl, 2 mM MgSO4, 100 μM CaCl2, and 2 g/L glucose at 30° C. For fatty acid supplementation experiments, sodium salts of a given fatty acid (Sigma-Aldrich) were added to a final concentration of 100 μg/mL. OD600 measurements were acquired every 15 min with a Bioscreen C Pro (Growth Curves USA) and plate was shaken continuously. Maximum specific growth rates were calculated using a spline model (https://github.com/scott-saunders/growth_curve_fitting).

Construction and screening of P. putida genomic library. A derivative of pBTL-2 (Prior et al., 2010) was constructed that lacked a BamHI restriction site (pKM037), which was further modified to contain a new BamHI site downstream of the pLac promoter (pKM062). pKM062 was subjected to a complete digestion with BamHI-HF (NEB), treatment with Antarctic Phosphatase (NEB), and the resulting linear fragment was isolated with the GeneJET Gel Extraction Kit (ThermoFisher Scientific). Genomic DNA from wild-type P. putida KT2440 was extracted using GeneJET Genomic DNA Purification Kit (ThermoFisher Scientific) and 32 μg was digested for 1 hr at 37° C. with 10 units of Sau3AI (NEB) followed by 20 min inactivation at 65° C. in a 1.6 mL reaction volume. The partially Sau3AI-digested genomic DNA was subjected to gel electrophoresis and fragments 2-6 kb were isolated with the GeneJET Gel Extraction Kit (ThermoFisher Scientific). Genomic DNA fragments (3.2 μg) were ligated to linearized pKM062 (450 ng) with T4 DNA ligase (NEB) for 16 hrs at 4° C. in a 200 uL reaction volume. An aliquot of the ligation mixture (5 μL) was transformed into 50 μL chemically competent NEB 5-α F′Iq E. coli 23 times and the resulting cells split between two LB agar plates supplemented with 50 μg/mL kanamycin, yielding 46 plates, and incubated at 37° C. overnight. Based on a dilution series, we estimated that each plate contained 1,350 colonies or about 62,000 colonies in total. Colony PCR screening of a sampling of isolates demonstrated that 5/21 transformants contained pKM062 without any insertion. The colonies on all 46 plates were collected with 1 mL of LB each and subjected to plasmid extraction with the GeneJET Plasmid Miniprep Kit (ThermoFisher Scientific). Isolated plasmids were pooled and vacuum-concentrated to 1 mL with a final concentration of 145 ng/μL.

To screen for rescue of the E. coli ΔfabH colony size defect, strain KM128 (E. coli MG1655 ΔfabH) was grown overnight at 37° C. and electrocompetent cells were prepared as previously described. Electrocompetent cells were mixed with 15 ng of the genomic library, electroporated, and recovered in 1 mL SOC Outgrowth Medium (NEB) shaking at 37° C. for 1 hr. Each transformation culture was distributed among 10 LB agar+50 μg/mL kanamycin plates (100 μL/plate) and grown at 37° C. overnight. Plates supplemented with 1 mM IPTG (GoldBio) were also utilized. Colonies substantially larger than the rest of the population were identified, subjected to plasmid extraction with the GeneJET Plasmid Miniprep Kit (ThermoFisher Scientific), and the plasmids were sequenced with oKM_0172 and oKM_0173 to determine the genomic fragment present.

Cell size determination by light microscopy. E. coli cultures were inoculated from glycerol stocks into 10 mL of LB Broth (Miller; Sigma-Aldrich) with 50 μg/mL kanamycin in a 125 mL flask and incubating overnight at 37° C. and 225 rpm. Overnight cultures were then used to inoculate a second seed culture into 10 mL of LB Broth (Miller; Sigma-Aldrich) with 50 μg/mL kanamycin in a 125 mL flask starting at an OD600=0.2 using a GENESYS™ 140 Visible Light Spectrophotometer (Thermo Scientific). The second seed cultures were incubated at 37° C. at 225 rpm until an OD600 of approximately 1 was reached. A 10 μL aliquot of each culture was applied to slides treated with 0.1% (w/v) poly-L-lysine to stabilize cells for imaging (Electron Microscopy Sciences). Optical polarized light microscopy was conducted using a Nikon E800 confocal microscope (Nikon). A total of 16 images were captured using a 100×1.4NA Plan Apo objective (Nikon) and a SPOT RTKE CCD camera (Diagnostic Instruments) for each strain. Actively dividing cells were manually curated, and the Analyze Particles functionality of the image processing software Fiji was used to calculate their area.

Gas chromatography-mass spectrometry analysis of fatty acid methyl esters. P. putida seed cultures were inoculated from glycerol stocks into 10 mL of LB Broth (Miller; Sigma-Aldrich) in a 125 mL flask and incubated overnight at 30° C. and 225 rpm. Overnight cultures were then used to inoculate a second seed culture into 20 mL of LB Broth (Miller; Sigma-Aldrich) in a 125 mL flask measured at OD600=0.2 using a GENESYS™ 140 Visible Light Spectrophotometer (Thermo Scientific). The second seed cultures were incubated at 30° C. at 225 rpm until an OD600 of approximately 1 was reached. The second seed cultures were pelleted and washed three times with 1× phosphate-buffered saline (137 mM NaCl, 10 mM phosphate, 2.7 mM KCl, pH 7.5). Cell pellets were resuspended in 100 μL of Nanopure™ water on ice and transferred to 1.8 mL borosilicate glass vials with PTFE-lined caps (DWK Life Sciences, Millville, NJ). Samples were frozen on dry ice and lyophilized overnight in a FreeZone 6 Plus (Labconco Corp.). Prior to esterification, 400 pg of benzoic acid (RING-D5) was added as an internal standard (Cambridge Isotope Laboratories, Inc.). Subsequently, 500 μL of 1.25 M hydrogen chloride-methanol solution (Sigma-Aldrich) was added to the samples and caps were sealed tightly. Vials were vortexed for 20 sec then centrifuged at 3,000 g for 3 min. Samples were incubated at 95° C. for 4 hrs while shaking at 600 rpm in an Eppendorf ThermoMixer, then allowed to cool at room temperature for 15 min. Then, 500 μL of Nanopure water and 500 μL of hexane (Sigma-Aldrich) were added to each vial. Sample vials were vortexed for 30 sec then centrifuged at 5,000 g for 10 min, and the upper hexane phase was transferred to a 2 mL glass autosampler vial (Microsolv Technology Corp.). An additional 500 μL of hexane was added to each sample vial, the vials were vortexed and centrifuged as before, and the hexane phase was again transferred to the autosampler vial. One hundred microliters of each sample were transferred to new autosampler vials equipped with 300 μL glass inserts for GC-MS analysis.

Samples were analyzed by an Agilent 8890 GC using a HP-5MS column (30 m×mm×0.25 μm; Agilent Technologies) coupled with a 5977B single quadrupole MSD (Agilent Technologies). A sample volume of 1 μL was injected into a splitless port with a constant inlet temperature of 250° C. The GC temperature was held at 60° C. for 1 minute following injection, then increased to 325° C. at a rate of 10° C. min-1 and held at 325° C. for 10 min. Fatty acid methyl esters were identified by comparison with authentic standard mixes (C8-28, Sigma-Aldrich; C8-C24, Restek Corp.), by matching experimental spectra with a FAMEs library, containing spectra and validated retention indices, and through matches with the NIST20/Wiley 11th GC-MS library. Peak areas were normalized for the internal standard and dry sample weight.

Protein production. Expression plasmids for producing MadB (pKM139), EcMadA (pKM162), holo-EcAcpP (pKM181), apo-EcAcpP (pKM175), EcFabD (pEUK070), EcFabH (pEUK069), Sfp (pET-Sfp, #159617 Addgene), FabH1 (pEUK078), and FabH2 (pEUK079) were transformed into E. coli BL211(DE3). LB Broth (Miller; Sigma-Aldrich) supplemented with 100 mg/mL ampicillin was used for the protein production. One liter of the main culture was grown in a 2.5 mL baffled flask, seeded with 5 mL of an overnight grown starter culture, and grown at 37° C. and 200 rpm. The culture was induced with 1 mM IPTG upon reaching OD600 ˜0.7 and the temperature was lowered to 18° C. The culture producing holo-EcAcpP was also supplemented with 0.5 mM D-pantothenate at the point of induction. The resulting biomass was harvested by centrifugation following about 16 hours incubation post induction and stored at −80° C. until further use.

Protein purification. Poly-His-tagged proteins (apo-AcpP, holo-AcpP, EcFabD, EcFabH, FabH1, FabH2, EcMadA) were purified using the combination of an immobilized metal affinity chromatography (IMAC) and anion exchange chromatography. MadB was tagless and purified using the combination of anion and cation exchange chromatographies. Sfp was purified following an established protocol (Yin et al., 2006). The ion exchange chromatography was performed using an ÄKTA fast protein liquid chromatography (FPLC) system (Cytiva). All purifications were performed at room temperature using 20 mM HEPES, 100 mM NaCl pH 7.5 (Buffer A) unless stated otherwise. Protein concentration was determined using Pierce™ Rapid Gold BCA Protein Assay Kit (Thermo Scientific). The frozen biomass was thawed, resuspended in minimal volume of Buffer A spiked with a trace (˜2 mg) of DNAseI, and lysed by sonication at 4° C. The lysate was cleared by centrifugation and a passage through 0.45 μm filter prior to purification by chromatography. Cleared lysate of a poly-His-tagged protein was applied to a HisTrap cartridge or a gravity-fed Ni Sepharose resin (Cytiva), washed with up to 20 mM imidazole, and eluted with 400 mM imidazole. The eluate was concentrated and exchanged to buffer A using a spin column with appropriate size cutoff. The protein was further purified using anion exchange chromatography (Source 15Q, Cytiva) and eluted using a linear NaCl gradient in Buffer A. Fractions of interest was pooled, concentrated, exchanged to buffer A using spin columns, frozen as beads over a liquid N2 bath, and stored at −80° C.

Cleared lysate of MadB was applied to an anion exchange resin (Source 15Q, Cytiva) using Buffer A. The flowthrough was collected and concentrated using a spin column. The protein concentrate was serially diluted to 20 mM citrate pH 5. Precipitated protein was removed by centrifugation and the soluble fraction was applied to a cation exchange resin (Source 15S, Cytiva). MadB was eluted with a linear NaCl gradient in 20 mM citrate pH 5. Fractions of interest was pooled, concentrated, exchanged to buffer A using spin columns, frozen as beads over liquid N2 bath, and stored at −80° C.

Size exclusion chromatography (SEC) analysis of MadB. The biologically relevant oligomeric structure of MadB was determined using HiLoad 16/600 Superdex 75 pg operated with an ÄKTA FPLC system (Cytiva) running isocratically at 0.85 mL/min in 20 mM Tris-C1, 100 mM NaCl pH 8 at room temperature. The sample injection was 500 μL of 1 mg/mL MadB diluted in the SEC running buffer. The retention time of MadB was compared to a series of standards (Cytiva): blue dextran (200 kDa), aldolase (158 kDa), conalbumin (75 kDa), ovalbumin (43 kDa), carbonic anhydrase (29 kDa), ribonuclease A (13.7 kDa), and aprotinin (6.5 kDa).

Acyl-EcAcpP production for MS analysis. Various acyl-EcAcpP was produced in one-pot in vitro reactions for MS-analysis in HEPES (I=0.1 M) pH 7.5 supplemented with 10 mM MgCl2 and 1 mM TCEP. The reaction was left at room temperature for at least 5 min after the last component was added. The mixture was flash frozen and stored at −20° C. until LC-MS analysis. EcAcpP, in either apo- or holo-forms, was added at 100 μM, acyl-CoA at 100 μM, and acyl-EcAcpP modifying enzymes at 5 μM.

The activity of FabH1 and FabH2 was evaluated in a mixture containing holo-EcAcpP (100 μM), EcFabD (5 μM), and malonyl-CoA (500 μM) to produce malonyl-EcAcpP. Subsequently, acetyl-CoA (500 μM), octanoyl-CoA (500 μM), and 5 μM of either FabH1 or FabH2 to produce the 3-ketoacyl-EcAcpP species. An equivalent control reaction (without octanoyl-CoA) to produce acetoacetyl-EcAcpP was made using EcFabH.

The decarboxylation of malonyl-EcAcpP by MadB or EcMadA was evaluated in a mixture containing holo-EcAcpP (100 μM), EcFabD (5 μM), malonyl-CoA (500 μM), and MadB (5 μM). Alternatively, the decarboxylation activity was evaluated in a mixture containing apo-EcAcpP (100 μM), Sfp (5 μM), malonyl-CoA (500 μM), and MadB (5 μM). The latter route was used as EcFabD can partially decarboxylate malonyl-EcAcpP to acetyl-EcAcpP; by contrast, Sfp did not appear to have a decarboxylative activity. Control reactions producing malonyl-EcAcpP and acetyl-EcAcpP, with an appropriate acyl-CoA donor, were made similarly employing acyl- and acyl-phosphopantetheine transferase activity of FabD and Sfp, respectively. As MadB was shown to decarboxylate malonyl-CoA, an alternate experimental setup was devised to differentiate whether the acetyl-EcAcpP was produced from the decarboxylation of malonyl-EcAcpP, or from Sfp- or FabD-catalyzed reaction between acetyl-CoA and EcAcpP. In this setup, a mixture producing malonyl-EcAcpP, by means of Sfp, was preincubated for 5 minutes to produce malonyl-EcAcpP prior to being split into three separate aliquots: MadB was added to the first aliquot, EcMadA to the second, and the last aliquot was left as a ‘no enzyme’ control. The extent of the decarboxylation activity was monitored as a function of malonyl-EcAcpP depletion relative to the ‘no enzyme’ control.

Experiments evaluating whether MadB can catalyze the Claisen-condensation reaction (akin to EcFabH) were performed in a mixture containing holo-EcAcpP (100 μM), EcFabD (5 μM), malonyl-CoA (500 μM), acetyl-CoA (500 μM), and MadB (5 μM). A control reaction producing acetoacetyl-EcAcpP was produced by substituting PP_0262 with EcFabH. Similarly, experiments evaluating whether MadB can catalyze a direct acyl-transferase reaction (akin to EcFabD) were performed in a mixture containing holo-EcAcpP (100 μM), acetyl-CoA (500 μM), and MadB (5 μM). A negative control reaction without MadB was also ran for comparison.

Transformation of non-EcAcpP substrate by MadB. Malonyl-CoA, methylmalonyl-CoA, and malonate were tested as potential non-EcAcpP-tethered substrates for MadB. The reaction contained 1 mM of the alternate substrate and 5 μM of MadB in HEPES (I=0.1 M) pH 7.5 supplemented with 10 mM MgCl2 and 1 mM TCEP. The reaction was incubated at room temperature with an intermittent agitation for −1 hour prior to being passed through a 0.2 μm filter and frozen until further analysis.

High-performance liquid chromatography (HPLC) and liquid chromatography mass spectrometry (LC-MS). Intact protein LC-MS was performed by diluting protein samples 500 times in 2% acetonitrile and 0.1% formic acid in water. Five microliters of the diluted solution of each sample were injected into the LC system. A Waters NanoAquity LC system equipped with two binary nanoflow pumps was used. Solvent A was 0.2% formic acid in water, and solvent B was 0.2% formic acid in acetonitrile. One pump was used for online desalting at 3 μL/min for 6 min using 10% solvent B with a short trapping column (in-house packed, 5 cm, inner diameter 150 μm, outer diameter 360 μm, C2 reversed phase, MEB2-3-300, Separation Methods Technologies). The other gradient pump was operated at 0.3 μL/min with an analytical column with C2 stationary phase (in-house packed, 50 cm, inner diameter 100 μm, outer diameter 360 μm, same packing material as the trap column). The separation gradient was from 10-40% solvent B over 25 min, followed by 5 min ramp to 60% B. A Thermo Orbitrap Exploris 480 was used for MS data collection, with data-dependent acquisition with higher energy collision dissociation (HCD). The raw data were summed over 14-24 min in retention time and deconvoluted using Intact Mass v 4.1 (ProteinMetrics). Charge vector spacing was 0.1, baseline radius was 15 m/z, smoothing sigma was 0.01 m/z, spacing was 0.005 m/z, mass smoothing sigma was 0.1, mass spacing was 0.05. The LC-MS data were deposited into MassIVE.ucsd.edu and can be accessed with identifier MassIVE accession: MSV000090294. We also examined intact masses of the same diluted protein samples by matrix assisted laser desorption/ionization (MALDI) using dried spot method as outlined in more detail below. The results were similar to LC-MS but contained more adducts due to lack of desalting and additional LC separation. Thus, the latter results are not reported here.

Detection of various CoA analytes in the samples were performed on an Agilent 1200 HPLC system (Agilent Technologies) equipped with an Agilent 6120 mass spectrometer detector (MS). Each sample and standard were injected at a volume of 2 μL on a Phenomenex Luna C18(2)-HST column 100A′, 25 μm, 2.0×100 mm column (Phenomenex). The column temperature was maintained at 45° C. and the buffers used to separate the analytes of interest was 0.1% formic acid in water (A)/0.1% formic acid in acetonitrile (B). A gradient program was used to separate the analytes of interest: (A)=100% and (B)=0% at time t=0; (A)=100% and (B)=0% at t=1 min; (A)=50% and (B)=50% at t=7.65 min; (A)=30% and (B)=70% at t=9.33 min; (A)=30% and (B)=70% at t=10.67 min; (A)=100% and (B)=0% at t=10.68 min; (A)=100% and (B)=0% at t=13 min. The flow rate was held constant at 0.50 mL/min resulting in a run time of 13 minutes. The MS system was setup in positive electrospray ionization mode with a gas temperature of 350° C., drying gas at 12 L/min, nebulizer pressure set to 35 psig, and a Vcap voltage of 3000v. A total of four different masses in SIM mode from the MS detector were used to determine the presence and or absence of each analyte of interest. A mass of 810.5 m/z (M+H)+ was used for analyte acetyl-CoA, 854.5 m/z (M+H)+ for malonyl-CoA, 868.5 m/z (M+H)+ for methylmalonyl-CoA, and 824.5 m/z (M+H)+ for propionyl-CoA. One standard from each analyte was used to determine the elution order and retention times of the compounds.

Detection of malonic and acetic acid was performed on an Agilent 1290 HPLC system (Agilent Technologies) equipped with an Agilent G7117A diode array detector (DAD). NREL's laboratory analytical procedure “Determination of Structural Carbohydrates and Lignin in Biomass” (Sluiter et al., 2012) HPLC method was used to detect the analytes of interest as outlined below. Each sample was injected at a volume of 20 μL onto an BioRad Aminex HPX-87H column 9 μm, 7.8×300 mm column (BioRad) at a column temperature of 55° C. A DAD detector was used at a wavelength of 210 nm to detect the analytes of interest. Compounds were separated utilizing an isocratic flow of 0.01 N H2SO¬4 in water at 0.6 mL/min for a total run time of 27 min. Acid standards of malonic, and acetic acid were purchased from Sigma Aldrich (Sigma-Aldrich). A one-point curve was used to establish retention times for the analytes of interest.

Matrix assisted laser desorption ionization (MALDI) MS. Protein samples were diluted 50 times in 2% acetonitrile with 0.1% formic acid. MALDI matrix was prepared by dissolving 2,5-dihydroxyacetophenone (15 mg/mL) in 90% acetonitrile with 0.2% trifluoroacetic acid. Protein samples were mixed with the MALDI matrix solution in a 1:1 ratio (v/v) and the mixture (1 μL) was deposited on a conductive indium tin oxide (ITO) glass slide. MALDI mass measurements were performed on ThermoFisher ultra-high mass range (UHMR)-Orbitrap coupled to a MALDI source (Spectroglyph, LLC) with a 1 kHz Explorer One Nd:YAG (349 nm) laser (Zemaitis et al., 2022). The instrument was operated in a positive mode over a m/z range of 6,000 to 20,000 with ˜40K resolving power at 10,000 m/z.

Protein crystallization. For crystallization, MadB was further purified with size-exclusion chromatography and eluted as a dimer into 20 mM Tris pH 8.0 and 150 mM NaCl. The protein was concentrated to 10 mg/mL and sitting drop co-crystallization trials were set up with a Mosquito crystallization robot (sptlabtech) using SWISSCI 3-lens low profile crystallization plates. Crystals grew in condition A5 (25% PEG 1500 and 0.1 M SPG buffer at pH 8.0) of the PACT screen (Molecular Dimensions). Crystals were cryo-protected with 20% glycerol and flash-frozen into liquid nitrogen. Diffraction data was collected on beamline 103 at the Diamond Light Source and automatically processed with STARANISO on ispyb. The structure was solved within CCP4 Cloud by molecular replacement with MOLREP using a search model created by the phyre2 server. Model building was performed in Coot and the structures was refined with REFMAC. MolProbity was used to evaluate the final model and PyMOL (Schrödinger, LLC) for protein model visualization. The atomic coordinates have been deposited in the Protein Data Bank and are available under the accession code 8AYV. Comparison of the MadB structure with the protein architectures of structural homologs was performed with the Dali server.

Benchmark of computational method. To validate the computational method used in the manuscript, we benchmarked the single point energies of the transition states and key intermediates presented in the manuscript with different combinations of density functional, basis set, and solvation models. Because all tested computational methods produced consistent results, suggesting the C3 carbonyl-stabilized pathway is the lowest energy pathway, we conclude that the computational method presented in the manuscript is reliable. Not only are the trends consistent within a certain intermediate despite the functional, the trends in energy change between two different intermediates are also consistent, indicating that the computed reaction mechanism in this study is accurate.

Scan of decarboxylation reaction coordinate without MadB. Scan of the decarboxylation reaction coordinate profile along the C—C distance helped us locating the decarboxylation transition state. We observe that the decarboxylation reaction coordinate is relatively flat, and we expect a transition state with C—C distance close to 2.50 Å.

Effects of single amino acid residues on decarboxylation transition state. We studied the effects of single amino acid residues on the decarboxylation transition state. We observed that N45 and H46 are stronger hydrogen bond donors that stabilize the anionic charge built up on the carbonyl oxygen atom in TS1.

Melting temperature determination by DSC. The apparent melting temperature values for wild-type MadB and the N45A and H46A mutants were assessed by DSC. Immediately prior to DSC analysis, to ensure both mono-dispersity and an optimal buffer match, each enzyme was prepared by SEC through a HiLoad Superdex 75 pg column (Cytiva) pre-equilibrated with the reference buffer comprising 100 mM NaCl, 50 mM NaH2PO4 pH 7.5 (NP75). The SEC column was calibrated with a mixture of globular protein standards (Sigma-Aldrich)—ribonuclease A (13.7 kDa), albumin (67.0 kDa), γ-globulin (158 kDa) and thyroglobulin (670 kDa)— to allow for the calculation of an apparent molecular weight for native MadB from its elution volume. Subsequently, triplicate DSC analyses, using 0.023 to 0.375 mg/mL wild-type enzyme (1.39 to 22.2 μM monomer concentration), were performed on a MicroCal PEAQ-DSC-Automated instrument (Malvern Panalytical). The two mutant enzymes were analyzed at 0.2 mg/mL. The sample and reference cells were raised in temperature from 30° C. to 120° C. at a rate of 1.5° C./min in low feedback mode.

Kinetic determination using ITC. Turnover of coenzyme A derivatives by each MadB variant was investigated using ITC on a MicroCal PEAQ-ITC-Automated instrument (Malvern Panalytical) utilizing the same NP75 buffer as above. All analyses were performed at 30° C. using high feedback mode. Stock solutions of acetyl-CoA and malonyl-CoA were prepared by dissolving the solid substrates (Sigma-Aldrich) in NP75 buffer and adjusting the pH to 7.5 with small aliquots of 1M NaOH, and then quantitated by UV absorbance spectroscopy using a molar extinction coefficient of 15,400 M-1 cm-1. Substrate preference and the apparent reaction enthalpy (ΔHapp) for malonyl-CoA turnover were assessed using a single-injection enzyme kinetics method (Todd and Gomez, 2001) with a reference power of 20 μcal/s and, following the injection, initial concentrations of 9.43 μM wild-type MadB and 371 μM substrate.

Subsequently, the kinetics of malonyl-CoA turnover was studied, in triplicate, using a multi-injection kinetics assay with a reference power of using 20 μcal/s. In each replicate, twelve low-volume aliquots of 47.7 mM malonyl-CoA were injected into the ITC cell containing either 1.00 μM wild-type MadB, 20.0 μM MadB (N45A) or 2.50 μM MadB (H46A) (monomer concentration) at 3 min intervals to give total substrate concentrations of up to 7.2 mM; higher malonyl-CoA concentrations could not be reached because of the large exothermic heats of dilution upon substrate injection. Power measurements were averaged over a window 150-180 s after each injection and converted to reaction rate using the determined ΔHapp of −3.56 kcal/mol (−14.9 kJ/mol). Data analysis and Michaelis-Menten curve fitting was performed using the instrument's analysis software (v1.40).

Results

P. putida strains lacking KASIII homologs are viable. In an effort to identify candidate KASIII enzymes in P. putida, we performed a BLAST search against the P. putida proteome using E. coli FabH (EcFabH, hereafter) as the query sequence. We identified two predicted proteins that we deemed FabH1 (PP_4379) and FabH2 (PP_4545), which exhibit 29% and 26% identity to EcFabH, respectively. Unlike the gene encoding EcFabH, FabH1 and FabH2 are not embedded within an operon that contains other fatty acid biosynthesis genes (FIG. 2A). Characteristic of known KASIII enzymes, however, FabH1 and FabH2 both contain the canonical catalytic triad of residues important for enzymatic function. To determine what role, if any, FabH1 and FabH2 play in fatty acid initiation in P. putida grown in a modified M9 media with glucose as the carbon source, we generated P. putida strains that lacked these corresponding genes both singly and in combination. Growth curve analyses of these deletion strains demonstrated that they did not exhibit a marked growth defect, but rather resembled wild type P. putida (FIG. 2B).

FabH1 and FabH2 exhibit different acyl-CoA substrate specificities. Considering the lack of overt phenotype observed in the double deletion strain, we were curious if either FabH1 or FabH2 exhibited any fatty acid initiation activity at all. To examine the in vivo activity of both proteins, we expressed them heterologously in a ΔfabH E. coli strain we generated using CRISPR-Cas9. While the ΔfabH E. coli strain displayed a reduced maximal growth rate compared to wild type, plasmid-based expression of EcfabH or fabH1 partially restored wild type-like growth in this genetic background (FIG. 2C). Expression of fabH2 did not improve the growth of the ΔfabH E. coli strain (FIG. 2C). This suggested that while FabH1 may have EcFabH-like activity, FabH2 may catalyze a different reaction. Indeed, FabH2 exhibits 75% amino acid sequence identity with a Pseudomonas aeruginosa protein (PA3286) that accepts octanoyl-CoA instead of acetyl-CoA.

We next validated the activity and specificity of the P. putida EcFabH homologs in vitro by determining their production of 3-ketoacyl-ACP species using a high mass accuracy intact protein liquid chromatography-mass spectrometry (LC-MS). In the following assays, malonyl-ACP was produced by EcFabD-catalyzed acyl transfer reaction between holo-ACP and malonyl-CoA. In the presence of malonyl-ACP and acetyl-CoA, purified EcFabH produced β-acetoacetyl-ACP (FIG. 2D; Reaction I). When supplied with malonyl-ACP and a 1:1 mixture of acetyl-CoA:octanoyl-CoA, purified FabH1 only performed the condensation reaction with acetyl-CoA and malonyl-ACP producing 3-acetoacetyl-ACP (FIG. 2D; Reaction II). In contrast, FabH2 specifically utilized octanoyl-CoA, producing 3-keto-decanoyl-ACP (FIG. 2D; Reaction III). The octanoyl-ACP was likely produced due to the promiscuous acyl-transfer activity of activity of FabD between acyl-ACP and octanoyl-CoA (Marcella and Barb, 2017). Overall, these in vitro activities are consistent with the ability and inability of FabH1 and FabH2, respectively, to rescue growth of the ΔfabH E. coli strain. In addition, gas chromatography-mass spectrometry analysis of fatty acid methyl esters (GC-MS-FAME) obtained from P. putida ΔfabH1 and ΔfabH2 strains was consistent with their demonstrated activities. Strains lacking FabH1 displayed a reduction in C14 and C16 species and a concomitant increase in C18 species, as previously seen in E. coli ΔfabH strains. Strains lacking FabH2 had a conspicuous accumulation of β-hydroxyoctanoic acid, not detected in either wild-type or ΔfabH1 strains, consistent with octanoyl-CoA entering the fatty acid degradation pathway rather than being shunted into fatty acid biosynthesis by FabH2. These data suggest that the viability of the ΔfabH1 ΔfabH2 double mutant is due to an additional KASIII-independent mechanism for fatty acid biosynthesis initiation. Additionally, these observations also provide a confirmation of FabH activity in P. putida.

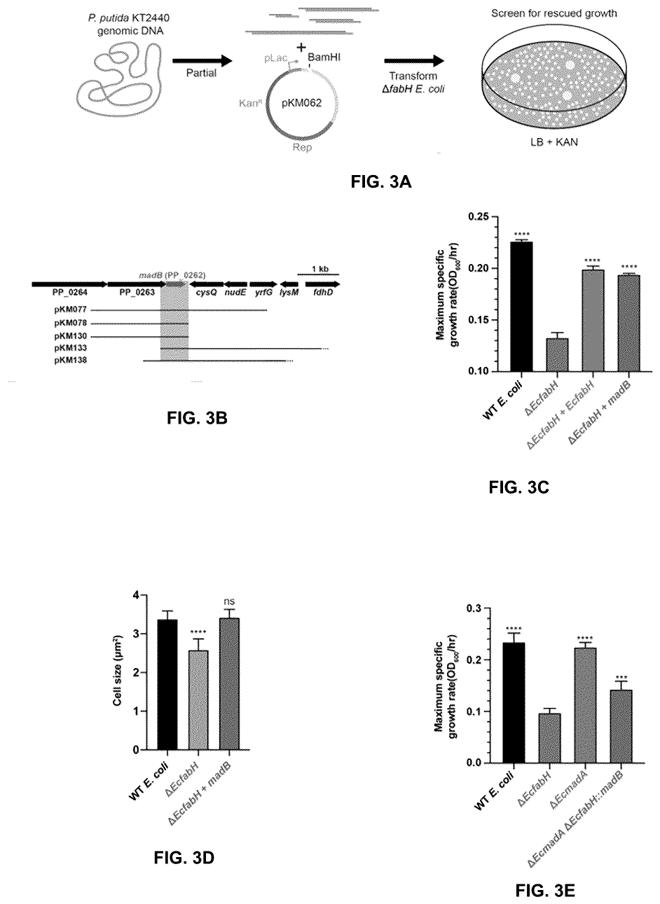

Expression of PP_0262 rescues E. coli ΔfabH growth and cell size defects. To elucidate other genes in P. putida with fatty acid initiation activity, we utilized a forward genetic screen (FIG. 3A), taking advantage of the small colony phenotype exhibited by ΔfabH E. coli strains. Briefly, we cloned P. putida genomic fragments into pKM062, a derivative of pBTL-2, generating a plasmid library. We then electroporated this plasmid library into a ΔfabH E. coli strain, selected for kanamycin-resistance, and visually screened for large colonies corresponding to rescue of the ΔEcfabH growth defect. Using this method, we isolated 52 large-colony transformants and subjected their respective plasmids to Sanger sequencing. Most of the recovered plasmids contained non-overlapping fragments of the P. putida genome. Indeed, several recovered plasmids contained no insert at all. Despite the high level of apparent false positives, we isolated five plasmids that all shared the P. putida genomic region containing PP_0262, which encodes a 151 amino acid protein that we hypothesized to be a MadB enzyme (FIG. 3B). To verify that madB alone can rescue the ΔfabH E. coli growth defect, we cloned the open reading frame of madB into an expression plasmid and introduced it into the ΔfabH E. coli strain. Heterologous expression of madB in the ΔEcfabH mutant restored wild type-like growth, comparable to the EcfabH control (FIG. 3C).

In addition to a small colony phenotype, E. coli strains lacking FabH have been reported to have reduced cell size. To determine if expression of madB can restore wild type-like cell size in a ΔEcfabH strain, we used microscopy to examine actively dividing cells from wild type, ΔEcfabH, and ΔEcfabH+madB strains. As previously reported, we confirmed ΔEcfabH cells are significantly smaller than wild-type cells (P<0.0001) (FIG. 3D). Expression of madB in ΔEcfabH cells, notably, rescued this small cell size defect (FIG. 3D). The ability of madB to restore both the growth and cell size defects of ΔfabH E. coli strains suggests madB has a role in fatty acid initiation.

MadB supports growth of E. coli lacking both native FabH and MadA. Although MadB appeared to have fatty acid initiation capabilities comparable to EcFabH, it remained a formal possibility that MadB does not have a direct role in fatty acid initiation. For example, E. coli strains lacking FabH can still grow, albeit poorly, due to the presence of MadA (YiiD). Furthermore, overexpression of madA has been demonstrated to rescue both the growth and cell size defects of ΔfabH E. coli strains. To rule out any indirect effects MadB may exert on madA expression, we generated an E. coli strain that lacked madA and had fabH replaced with madB using CRISPR-Cas9 (FIG. 3E). The rescue of the synthetically lethal ΔfabH ΔmadA E. coli strain with madB supports a direct role for MadB in fatty acid initiation.

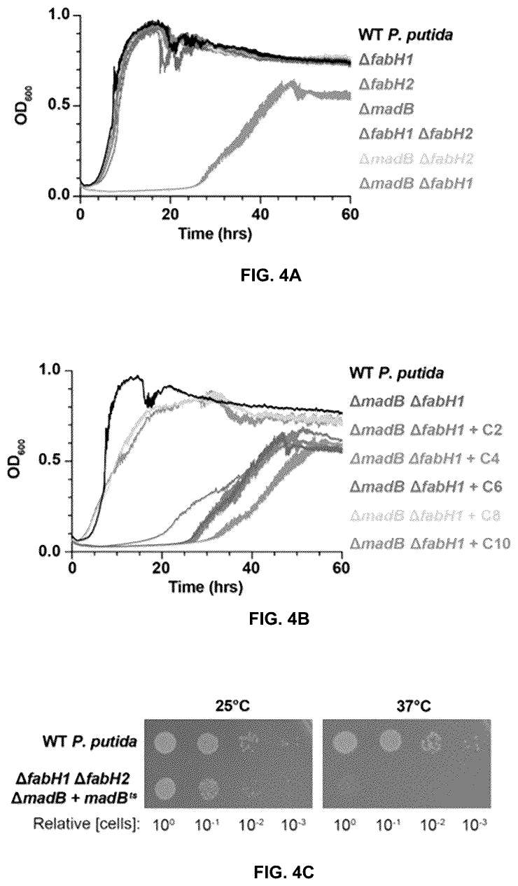

Synthetic growth defect of ΔmadB ΔfabH1 P. putida is rescued by medium-chain-length fatty acids. To examine the role of MadB in P. putida fatty acid initiation, we generated strains lacking madB singly and in combination with ΔfabH1 and ΔfabH2. We found that all single and double mutants, except ΔmadB ΔfabH1, displayed wild type-like growth (FIG. 4A). This phenotype contrasts ΔfabH E. coli, with an intact madA, that displayed a stunted growth rate (FIG. 3E) and diminished colony size. The P. putida strain lacking both MadB and FabH1 exhibited a pronounced lag-phase and a reduced final optical density (FIG. 4A). GC-FAME of a ΔmadB single mutant, however, did not display overt fatty acid biosynthesis defects like the mutants lacking fabH1. These data suggest that MadB and FabH1 are redundant in maintaining a wild type-like growth rate, but FabH1 contributes more to wild-type fatty acid biosynthesis.

Since the ΔmadB ΔfabH1 double mutant still retained FabH2, which we demonstrated can accept a medium-chain-length acyl-CoA (FIG. 2D; Reaction III), we surmised that supplementation of the minimal growth medium with medium-chain-length fatty acids may rescue the growth defect. Indeed, we observed a dramatic improvement of growth when ΔmadB ΔfabH1 was supplemented with C8 and C10 straight-chain saturated fatty acids (FIG. 4B). Supplementation with C6 also appeared to improve growth, albeit less significantly, while C4 and C2 inhibited and had no effect on growth, respectively (FIG. 4B). This in vivo evidence supports the specificity of FabH2 towards medium-chain-length acyl-CoA substrates.

Loss of FabH1, FabH2, and MadB is synthetically lethal. To determine if we had identified all the fatty acid initiation enzymes in P. putida, we attempted to generate a P. putida strain lacking fabH1, fabH2, and madB. These initial efforts were unsuccessful. To test the hypothesis of synthetic lethality more definitively, we generated a P. putida strain lacking fabH1, fabH2, and madB, but containing a temperature-sensitive plasmid that expressed madB. Whereas wild-type P. putida showed similar growth at 25° C. and 37° C., the triple mutant bearing the temperature-sensitive plasmid displayed a dramatic loss of viability at the restrictive temperature (FIG. 4C). We did, however, observe some growth at higher cell densities for the triple mutant strain (FIG. 4C). We reasoned that these rare cells may have retained the plasmid bearing madB, even at the restrictive temperature of 37° C. Indeed, all triple mutant isolates capable of growing at 37° C. retained resistance to apramycin (35/35 colonies), which is conferred by the temperature-sensitive plasmid. These data suggest that triple mutants lacking FabH1, FabH2, and MadB are not viable and that these proteins constitute the entirety of fatty acid initiation enzymes in P. putida.

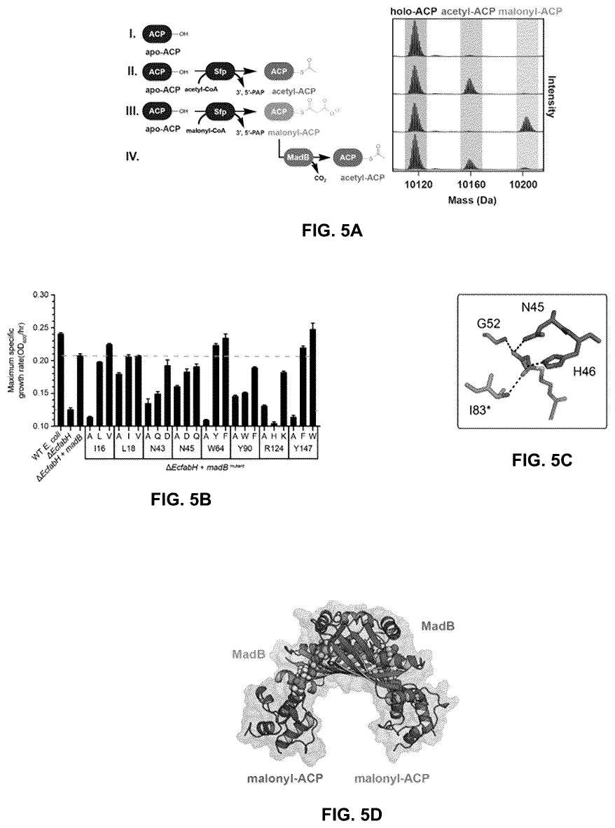

MadB is a malonyl-ACP decarboxylase. There are three potential routes to generate β-acetoacetyl-ACP for the initiation of fatty acid biosynthesis. The first route is the direct Claisen-condensation of acetyl-CoA with malonyl-ACP to yield β-acetoacetyl-ACP. We have shown that FabH1 is capable of this activity (FIG. 2D; Reaction II). The second route begins with the transfer of the acetyl group from acetyl-CoA onto holo-ACP, yielding acetyl-ACP. This acetyl-ACP can be subsequently condensed with malonyl-ACP, by either FabB/F, resulting in β-acetoacetyl-ACP. The third route similarly generates the acetyl-ACP precursor but via decarboxylation of malonyl-ACP. To determine which activity, if any, MadB exhibits, we purified MadB and monitored its ability to generate relevant ACP species in vitro by LC-MS. We found that MadB failed to catalyze either the Claisen-condensation reaction or the acetyltransferase reaction. However, addition of MadB to a solution containing malonyl-ACP resulted in a depletion of malonyl-ACP and a concomitant increase in acetyl-ACP (FIG. 5A). We also found that MadB is capable of decarboxylating malonyl-CoA and methylmalonyl-CoA to produce acetyl-CoA and propionyl-CoA, respectively, but not free malonate, thereby highlighting the importance of a pantetheine arm and the flexibility of MadB to accept a minor substitution along the malonyl group.

The nature of MadB-catalyzed decarboxylation of malonyl-CoA was also investigated using ITC. The mixing of malonyl-CoA and MadB emitted a sustained heat indicating an exothermic reaction (ΔHapp=−14.9 kJ/mol). In contrast, no heat was detected upon injection of acetyl-CoA, other than the transient heat of dilution due to the injection event. The kinetics of this reaction were investigated by a multi-injection ITC method, which revealed a Km of 12±1 mM and kcat of 1.4±0.1 s-1 for malonyl-CoA at 30° C., a catalytic efficiency that is likely too low to be physiologically relevant. Hence, MadB was appropriately deemed a malonyl-ACP decarboxylase in agreement with prior determination of MadA from E. coli and MadB from Shewanella oneidensis.

Alanine-scanning mutagenesis of MadB indicates residues important for function. To define the amino acids essential for MadB activity and gain insight into the reaction mechanism, we screened a complete library of variants harboring MadB alanine-substituted mutants for their inability to rescue the colony size defect of a ΔfabH E. coli strain, ultimately identifying eight such MadB variants. We subsequently verified the growth defect of these variants using a microplate reader (FIG. 5B). In addition, we investigated the consequence of more conservative substitutions at these eight sites identified by the alanine scanning screen (Ile16, Leu18, Asn43, Asn45, Trp64, Tyr90, Arg124, and Tyr147). In general, the more conservative substitutions were less detrimental when compared to their alanine counterpart, and some substitutions even appeared to outperform wild-type MadB suggesting opportunities for further engineering. (FIG.

MadB belongs to the hotdog fold protein family. To better understand the mechanism of MadB-catalyzed malonyl-ACP decarboxylation, we solved the structure of MadB via X-ray crystallography at 1.04 Å resolution (PDB 8AYV). MadB was structurally closest to a homolog from Chlorobaculum tepidum (PDB 3LMB; 38% sequence identity with a root-mean-square deviation (RMSD) of 1.6 Å). We found that MadB adopts a HDF configuration, which is common among enzymes involved in fatty acid metabolism. MadB exists as a dimer and this arrangement was observed in crystallography and corroborated by size-exclusion chromatography.

Differential scanning calorimetry analysis of MadB at 22.2 μM (monomer concentration) revealed that the enzyme thermally unfolds with a single transition at 66.4° C.; the lack of a second peak indicates that there is no significant monomer-dimer exchange at this concentration. This melting temperature remains constant upon two-fold dilution to 11.1 μM and 5.55 μM monomer.

Next, we sought to generate a model of MadB in complex with malonyl-ACP. We first performed a structural similarity search and found various structures of LnmK (4% sequence identity to MadB with RMSD of 3.0 Å) in substrate analog-bound complexes. LnmK is a previously characterized double-HDF enzyme that is a bifunctional decarboxylase/acyltransferase associated with the biosynthesis of leinamycin, a potential anti-cancer drug. The similar scaffold (HDF), substrate (methylmalonyl-CoA vs. malonyl-ACP), and activity (decarboxylation) of LnmK and MadB guided our efforts to first dock malonyl-CoA into the presumed active site of MadB. An initial model (Minitial) was built via structural alignment of MadB with LnmK in complex with 2-nitronate-propionyl-CoA (PDB 6X7L), which was easily modified in silico to generate the malonyl-CoA ligand. We refined Minitial with a molecular dynamics (MD) protocol that reproduces an induced-fit process, and generated two top models, MMD1_CoA and MMD2_CoA, with AutoDock Vina estimated binding affinities at −13.9 and −13.6 kcal/mol, respectively. Unlike MMD1_CoA, MMD2_CoA implicated residues AsnN45 and R124Arg124, identified in the alanine-scanning mutagenesis screen, as interacting with malonyl-CoA at its reaction center. In addition, MMD2_CoA established a hydrogen bond network with the terminal malonyl group analogous to that observed in LnmK (FIG. 5C. Together, these data suggest that MMD2_CoA captures legitimate interactions between MadB and malonyl-CoA.

We further performed MD-based refinement to generate a model of MadBmalonyl-ACP, using Minitial after the replacement of CoA with ACP from E. coli (PDB 4KEH; 86% identity to PP_1915 gene product, ACP from P. putida). Five conformations selected from the MD simulations suggested that a binding mode like MMD2_CoA is prevalent in the presence of ACP (FIG. 5D). Typical of other ACP-interacting enzymes, MadB exhibited a remarkable complementarity in charge and hydropathicity where it contacted helix II of ACP, also referred to as the “recognition helix”. This interface between MadB and ACP is spontaneously formed in our simulations, indicating the robustness of the model.

Among the functional residues identified via alanine-scanning, our model of MadBmalonyl-ACP seems to explain the importance of residues Asn45 and Arg124 as being directly involved in substrate binding. Specifically, Asn45 and Arg124 bind the terminal carboxyl group of the malonyl and the pantetheine moieties, respectively. In addition, the identification of aromatic residues Trp64, Tyr90, and Tyr147 is likely explained by their role in forming the hydrophobic core underlying the substrate binding site. The remaining identified residues, Ile16, Leu18, and Asn43, are located near the dimer interface where the proposed decarboxylation occurs. To determine if these residues play an outsized role in MadB dimerization, we used the machine learning-based predictor KFC2a, which predicts residues that account for the majority of the binding affinity in a complex. We found that Ile16, Leu18, and Asn43, were among the eight residues predicted by KFC2a to be important for MadB dimerization. The independent identification and correlation of crucial residues in MadB through in vivo and in silico analyses strengthens the validity of our structural binding model.

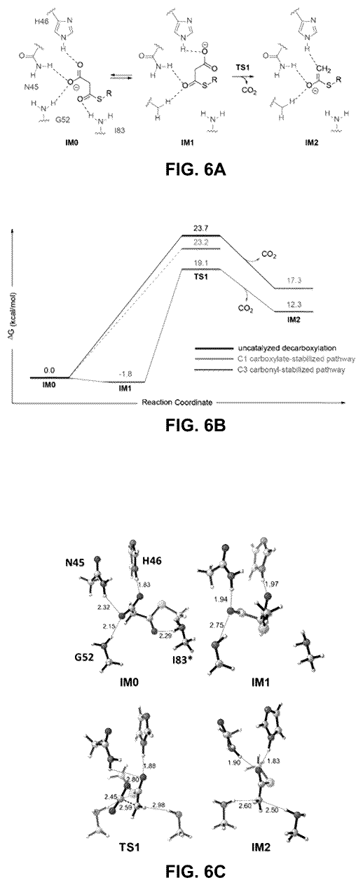

MadB catalyzes malonyl decarboxylation through carbonyl stabilization. Based on the precedence of the catalytic mechanism proposed for LnmK and the data presented in this study, we propose a catalytic mechanism for MadB (FIG. 6A). This proposed mechanism is equivalent to the first-half reaction of LnmK. Based on the MD docking results, we created a theozyme with Asn45, His46, Gly52, and Ile83. In IM0, hydrogen bonds with the Gly52 backbone and the N45 side chain stabilize the anionic charge of the terminal carboxylate of malonyl-ACP, while the Ile83 backbone stabilizes the carbonyl group. Alternatively, IM1 represents a different substrate orientation, in which hydrogen bonds with Gly52 and Asn45 stabilize the carbonyl, and the His46 stabilizes the carboxyl group of the intermediate. The lone-pair electrons are subsequently delocalized from the terminal carboxylate towards the f3-keto site, resulting in C—C bond fragmentation (decarboxylation) and the formation of an enolate intermediate (IM2). The anionic enolate in IM2 is then stabilized by the hydrogen bonds with Gly52, Asn45, and His46. By modeling effects of each amino acid residue independently, we identify the amide N—H in Asn45 and the imidazole N—H in His46 as strong hydrogen bond donors that can best stabilize an anionic substrate. Finally, deprotonation of IM2 will then readily produce the acetyl-ACP product. It is unclear whether the final resolution of the enolate would occur spontaneously via a proton exchange with a solvent species or whether the process is assisted by a density functional theory (DFT) protein side-chain.

From the terminal carboxylate of malonyl-ACP (IM0), we modeled the reaction coordinate profile to form the enolate intermediate IM2 through the decarboxylation transition state TS1 (FIG. 6B). Our calculations indicated that without MadB present, the uncatalyzed decarboxylation (FIG. 6B, black) transition state has a 23.7 kcal/mol free energy of activation, which suggests that the reaction is likely to proceed slowly at room temperature. Next, we considered two possible pathways by which the amino acids (Gly52, Ile83, Asn45, and His46) can stabilize the decarboxylation transition state (TS1). These two catalyzed pathways are differentiated on whether the substrate undergoes rearrangement in the enzyme active to form a more stable orientation (IM1). Additionally, the two catalyzed pathways are also distinguished by the role of Asn45 on whether it stabilizes the C1 carboxyl (FIG. 6B, orange) or the C3 carbonyl groups (FIG. 6B, blue), of the substrate in the transition state (TS1). The transition state in the C1 carboxyl-stabilized pathway, (FIG. 6B, orange), has a kinetic barrier of 23.2 kcal/mol which is only slightly lower than the uncatalyzed reaction indicating a minor contribution of the amino acid residues in facilitating the decarboxylation. Alternatively, MadB can more effectively promote decarboxylation along the C3 carbonyl-stabilized pathway (FIG. 6B, blue), as the transition state kinetic barrier was lowered to 19.1 kcal/mol. Closer evaluations of the DFT-optimized geometries along the pathways further highlight the effects of Asn45 and His46 in promoting the decarboxylation reaction (FIG. 6C). In the resting state (IM0), the anionic carboxylate group is stabilized by hydrogen bonding with Asn45 and Gly52, while the imidazole sidechain of His46 may interact with either the carboxylate or the carbonyl group of the substrate. In the transition state (TS1) of the C3 carbonyl-stabilized pathway, both the amide N—H in Asn45 and the imidazole N—H in His46 form hydrogen bonds with the carbonyl to stabilize the development of negative charge on the oxygen, a feature that is missing in the C1 carboxylate-stabilized pathway.

To further evaluate the relative contribution of amino acid side chains of Asn45 and His46 towards catalysis, we purified Asn45A and His46A variants of MadB and compared their catalytic efficiency for malonyl-CoA decarboxylation against the wild-type counterpart using the same ITC-based assay. This in vitro experiment provides a clearer phenotype when compared to the prior in vivo alanine scan experiment as described previously. Similar analyses, however, are not available for Gly52 and Ile83 as their hydrogen bonding interactions are contributed by the amide main chain. Both variants were purified similarly to the wild-type MadB. As described above, the MadB-catalyzed decarboxylation of malonyl-CoA is not physiologically relevant; however, this scheme is useful for comparing the performance of the different variants. The kinetic results are summarized in Table 1. While both variants have about half the Michaelis constant, their lowered turnover numbers dramatically affected the overall catalytic efficiency with Asn45Ala and His46Ala resulting in 13- and 1.5-fold lower catalytic efficiency, respectively when compared to the wild-type enzyme and are in line with our prior in vivo alanine scan result. The dramatic loss in turnover number in Asn45Ala variant is consistent with its purported role for transition state stabilization. Conversely, the less severe defect observed for His46Ala may stem from a less productive orientation of the bound substrate. Overall, along with the MD-derived model, our DFT calculations highlight the importance of multiple amino acids including Asn45, His46, Gly52, and Ile83 in promoting the decarboxylation reaction of the terminal carboxylate of malonyl-ACP at MadB.

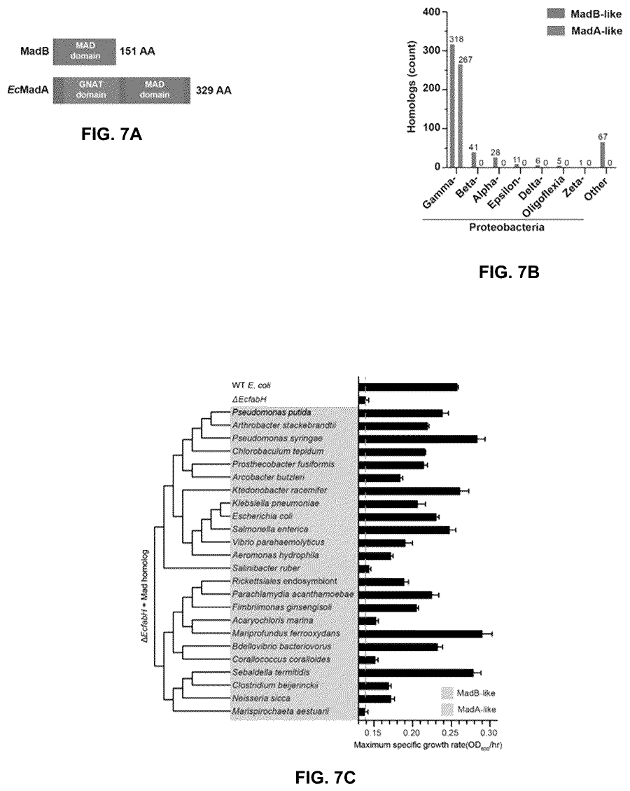

Functional homologs of MadB present in a diversity of bacterial lineages. To determine the phylogenetic distribution of MadB homologs, we utilized an iterative profile-HMM search method, JackHMMER, using MadB as the search query. We identified a total of 479 potential homologs with the same protein domain structure as MadB (FIGS. 7A, 7B). In addition, we found 267 potential homologs containing a predicted GNAT domain appended to the N-terminus (FIGS. 7A, 7B). One such dual-domain homolog is MadA from E. coli (EcMadA, hereafter FIG. 7A). We found that MadA-like proteins were restricted to Gammaproteobacteria, whereas MadB-like proteins were detected in 14 distinct phyla (FIG. 7B). To test if the homologs were functional, we surveyed the ability of 23 MadA/MadB-like proteins across 13 phyla for their ability to rescue the E. coli ΔfabH growth defect (FIG. 7C). We found that most of the examined homologs, both MadA- and MadB-like, conferred a growth advantage to ΔfabH E. coli (FIG. 7C). This suggests that MadA/MadB-like proteins can initiate fatty acid biosynthesis similarly in a multitude of bacteria species.

Discussion & Conclusions

Genetically dissecting the components of bacterial fatty acid biosynthesis has been traditionally difficult, in part, due to the essential nature of this process. For example, supplementing E. coli growth medium with fatty acids does not guarantee the viability of a particular fab mutant. Aided by genetic redundancy, here we identified and characterized the three enzymes, and three respective pathways, that initiate fatty acid biosynthesis in P. putida KT2440 (FIG. 1). Rather than being idiosyncratic to P. putida, these findings illuminate alternate fatty acid initiation in a wide variety of bacteria.

Our bioinformatics-driven approach identified FabH1 and FabH2 in P. putida KT2440 as potential KASIII enzymes. We found that FabH1, like EcFabH, catalyzes the Claisen condensation of acetyl-CoA with malonyl-ACP to produce β-acetoacetyl-ACP in vitro. In addition, P. putida ΔfabH1 strains display a similar fatty acid biosynthesis defect observed in an E. coli ΔfabH strain. For this reason, fabH1 appears to be the gene conspicuously absent from the P. putida “fabHDG” operon. Sequence analysis of FabH2 revealed high identity with a P. aeruginosa protein, PA3286 gene product. Like PA3286 gene product, FabH2 prefers octanoyl-CoA over acetyl-CoA, and produces β-keto-decanoyl-ACP. In wild-type P. putida, FabH2 likely helps assimilate exogenous fatty acids and recycle endogenous fatty acids. Interestingly, a strain relying on FabH2 alone for growth (ΔmadB ΔfabH1) is viable in minimal medium, suggesting sufficient side-activity with acetyl-CoA to support de novo fatty acid initiation.

Despite the apparent ability of FabH1 and FabH2 to initiate fatty acid biosynthesis, we found that they were unnecessary for P. putida viability due to the presence of madB, which we identified in a forward genetic screen. Loss of MadB alone does not cause an overt defect in fatty acid biosynthesis, unlike loss of FabH1, but the ΔmadB ΔfabH1 double mutant exhibits a severe growth defect. This suggests that FabH1 and MadB to a lesser degree, are the main initiation factors in P. putida. We interrogated three possible routes for MadB-catalyzed fatty acid initiation and discovered that MadB is capable of malonyl-ACP decarboxylation. The acetyl-ACP species produced by MadB is presumably condensed with malonyl-ACP by FabB/F, generating β-acetoacetyl-ACP. Whereas the FabH1 pathway only requires one malonyl-ACP and one acetyl-CoA to produce β-acetoacetyl-ACP, the MadB pathway requires two malonyl-ACP molecules; one to generate acetyl-ACP and another for the condensation reaction with FabB/F. This additional malonyl-ACP molecule, ultimately derived from acetyl-CoA, comes at the cost of ATP hydrolysis (FIG. 1A). This extra metabolic burden imposed by the MadB pathway may explain why the FabH1 pathway is the predominant initiation route. In E. coli, FabH similarly plays an outsized role in fatty acid initiation, whereas MadA is only employed under stress conditions. Future efforts in P. putida may elucidate whether comparable transcriptional regulation occurs with madB.

The decarboxylation of a malonyl group can occur through both abiotic and biotic processes. The non-enzymatic process requires a highly acidic condition and is initiated by the f3-carbonyl group abstracting a proton from the terminal carboxylate (pKa ˜0.3). Alternatively, an uncatalyzed reaction at neutral pH would encounter such a high activation energy barrier that the process would proceed at a glacial pace. By contrast, the MadB-catalyzed reaction enables FabH-independent FASII initiation such that no apparent growth defect is observed in P. putida ΔfabH1 ΔfabH2 strains. In addition to HDF, there are at least three additional enzyme scaffolds that have evolved means to catalyze the decarboxylation of a malonyl group at ambient conditions: crotonase, GNAT, and the biotin dependent Na+ translocating protein families; all but the last protein family are cofactorless systems. Specifically for the GNAT protein family, its counterpart in MadA is non-functional and its function remains unknown. Despite the distinct protein scaffolds, the active site architectures of the cofactorless systems are strikingly similar, employing elements for substrate polarization, often with the amide backbones highlighting a common catabolic logic: the polarization of the terminal carboxylate and a resonance stabilized f3-keto-enolate intermediate.

Unlike the first half of the malonyl decarboxylation, the final resolution of the enolate intermediate (IM2) remains elusive. It is unclear whether this process occurs spontaneously, through a proton exchange with a solvent species, or is assisted by an amino acid side chain. In the case of both methylmalonyl-CoA decarboxylase (MMCD, crotonase family) and LnmK (HDF family) a flexible acid catalyst was proposed facilitate this process; but this residue is not conserved, or its equivalent is missing in other enzyme systems indicating an alternate means for the keto-enol tautomerization to proceed. In MadB, this final acid catalyst role may be fulfilled by His80 as suggested by the predicted models of malonyl-derivatives in complex with MadB. Instead, His80 may possess additional role on an earlier stage of the reaction by attracting the electron density from the terminal carboxylate towards the β-keto carbonyl group. Nevertheless, other adjacent basic amino acids, such as His46 and Lys47, may at least partially compensate for the substitution His80Ala, explaining the non-essential role of His80 for catalysis.

The analyses provided by the structural modeling and the DFT calculation corroborates the function of the residues identified via the in vivo alanine scanning; namely, the direct interaction with the reaction core (Asn45) or with distal regions of the substrate (Arg124), the stabilization of the dimer assembly of the substrate-binding site (Ile16, Leu18, and Asn43), or the formation of its underlying hydrophobic core (Trp64, Tyr90, and Tyr147). Other residues that are key for catalysis according to the proposed reaction mechanism (Gly52 and Ile83) were not identified as functional residues via mutagenesis because, according to our models, their interactions with the substrate involve backbone atoms. Indeed, many of the initial substitutions in the alanine scan did not result in a complete growth defect in the ΔfabH E. coli background, likely due to the complex interplay between the mutant MadB and EcMadA. The result was only confounded as the amino acid residues involved in catalysis serve to polarize the substrate instead of acting as a direct proton shuttle. As such, the substitution effect would be dampened for example through the effect of ordered water or chemical rescue. Additionally, other structural features such as helix II of ACP (“recognition helix”) may play an important role in stabilizing the substrate in a reactive conformation, which may contribute to the preferential activity of MadB toward malonyl-ACP instead of malonyl-CoA through the complementary hydropathicity.

In summary, our study describes three routes for FASII initiation in P. putida KT2440. FabH1 and FabH2 are responsible for the conventional Claisen condensation type reactions utilizing short and medium chain acyl-CoA donors, respectively. The third route utilizes the malonyl-ACP decarboxylase system, MadB. Understanding these enzymes opens an avenue for tunability of fatty acid metabolism in P. putida and provides insight toward the development of a more efficient biocatalyst such as in the production of fatty-acid derived bioproducts. For example, carbon flux may be redirected to produce non-native fatty acid products by employing FabH homologs with different substrate specificities. Moreover, the prevalence of malonyl-ACP decarboxylase system in the domain Bacteria also opens an avenue for biomedical applications such as in the development of novel antibiotics target.

The foregoing disclosure has been set forth merely to illustrate the invention and is not intended to be limiting.

We claim:

1. A non-naturally occurring Pseudomonas sp. comprising a non-naturally occurring gene encoding for malonyl-ACP decarboxylase (MadB) wherein the gene (madB) has greater than 70% sequence identity to the nucleotide sequence of PP_0262 from P. putida KT 2440.

2. The non-naturally occurring Pseudomonas sp. of claim 1 wherein the Pseudomonas sp. is P. putida KT 2440.

3. A method for initiating fatty acid biosynthesis in P. putida sp. comprising overexpressing a non-naturally occurring gene encoding for malonyl-ACP decarboxylase (MadB) wherein the gene (madB) has greater than 70% sequence identity to the nucleotide sequence of PP_0262 from P. putida KT 2440.

4. A non-naturally occurring Pseudomonas sp. useful for the production of branched-chain polyhydroxyalkanoates, branched-chain 3-hydroxyacids (BCHA) (PHA monomers), and branched-chain fatty acids (BCFA) comprising an overexpressed gene selected from the group consisting of ₛfabH2, BKD (lpdV-bkdAA-bkd-AB-bkdB), lplA, fadR, phaC, phaG, alkK, acc, alsS-ilvCD, and leuAmodBCD.

5. The non-naturally occurring Pseudomonas sp. of claim 4 wherein the Pseudomonas sp. is P. putida KT 2440.

6. The non-naturally occurring Pseudomonas sp. of claim 4 wherein the gene is ₛfabH2.

7. The non-naturally occurring Pseudomonas sp. of claim 4 wherein the gene is BKD (lpdV-bkdAA-bkd-AB-bkdB).

8. The non-naturally occurring Pseudomonas sp. of claim 4 wherein the gene is lplA.

9. The non-naturally occurring Pseudomonas sp. of claim 4 wherein the gene is fadR.

10. The non-naturally occurring Pseudomonas sp. of claim 4 wherein the gene is phaC.

11. The non-naturally occurring Pseudomonas sp. of claim 4 wherein the gene is phaG.

12. The non-naturally occurring Pseudomonas sp. of claim 4 wherein the gene is alkK.

13. The non-naturally occurring Pseudomonas sp. of claim 4 wherein the gene is acc.

14. The non-naturally occurring Pseudomonas sp. of claim 4 wherein the gene is alsS-ilvCD.

15. The non-naturally occurring Pseudomonas sp. of claim 4 wherein the gene is leuAmodBCD.


SEQUENCE LISTING (97 sequences)

SEQ ID NO: 1 - DNA, 47 bp, synthetic construct
  source 1..47: mol_type = other DNA
acgcctcacacaggaaacagctatgactacgcagcgctctggaaaag

SEQ ID NO: 2 - DNA, 48 bp, synthetic construct
  source 1..48: mol_type = other DNA
gcttttaggggctgactgggatcatctggcttcccaatgaaaacagcc

SEQ ID NO: 3 - DNA, 48 bp, synthetic construct
  source 1..48: mol_type = other DNA
ggctgttttcattgggaagccagatgatcccagtcagcccctaaaagc

SEQ ID NO: 4 - DNA, 42 bp, synthetic construct
  source 1..42: mol_type = other DNA
acgttgtaaaacgacggccagtgccatcgagcagattacggc

SEQ ID NO: 5 - DNA, 20 bp, synthetic construct
  source 1..20: mol_type = other DNA
caccagcgacaccgagtatt

SEQ ID NO: 6 - DNA, 20 bp, synthetic construct
  source 1..20: mol_type = other DNA
caccgatggtcggcttatca

SEQ ID NO: 7 - DNA, 47 bp, synthetic construct
  source 1..47: mol_type = other DNA
acgcctcacacaggaaacagctatgactccttgaaggcgaacaaccg

SEQ ID NO: 8 - DNA, 20 bp, synthetic construct
  source 1..20: mol_type = other DNA
ctgagctaccacttcgagcc

SEQ ID NO: 9 - DNA, 20 bp, synthetic construct
  source 1..20: mol_type = other DNA
ctgagctaccacttcgagcc

SEQ ID NO: 10 - DNA, 20 bp, synthetic construct
  source 1..20: mol_type = other DNA
acaccctgaaggacaacgtc

SEQ ID NO: 11 - DNA, 24 bp, synthetic construct
  source 1..24: mol_type = other DNA
agggagaaaggcggacaggtatcc

SEQ ID NO: 12 - DNA, 24 bp, synthetic construct
  source 1..24: mol_type = other DNA
cggagaacctgcgtgcaatccatc

SEQ ID NO: 13 - DNA, 18 bp, synthetic construct
  source 1..18: mol_type = other DNA
atagtcctgtcgggtttc

SEQ ID NO: 14 - DNA, 20 bp, synthetic construct
  source 1..20: mol_type = other DNA
ccatcttgttcaatcatgcg

SEQ ID NO: 15 - DNA, 47 bp, synthetic construct
  source 1..47: mol_type = other DNA
acgcctcacacaggaaacagctatgacagttcgaatttgccaacggc

SEQ ID NO: 16 - DNA, 47 bp, synthetic construct
  source 1..47: mol_type = other DNA
aaggccgcgttagtcgtactataacggttgtcctcttgatcgtcatg

SEQ ID NO: 17 - DNA, 47 bp, synthetic construct
  source 1..47: mol_type = other DNA
catgacgatcaagaggacaaccgttatagtacgactaacgcggcctt

SEQ ID NO: 18 - DNA, 42 bp, synthetic construct
  source 1..42: mol_type = other DNA
acgttgtaaaacgacggccagtattgccggcagtgaagagtt

SEQ ID NO: 19 - DNA, 20 bp, synthetic construct
  source 1..20: mol_type = other DNA
gttgattctgggcgaaaccg

SEQ ID NO: 20 - DNA, 20 bp, synthetic construct
  source 1..20: mol_type = other DNA
ccgggagctcaaggaagaag

SEQ ID NO: 21 - DNA, 19 bp, synthetic construct
  source 1..19: mol_type = other DNA
gcgcgggggtaaacgtatg

SEQ ID NO: 22 - DNA, 20 bp, synthetic construct
  source 1..20: mol_type = other DNA
ctgggtgctgcctacctgat

SEQ ID NO: 23 - DNA, 45 bp, synthetic construct
  source 1..45: mol_type = other DNA
cgtaacaggcgcctgatgaatcaggtcgttcctctggtcaaaggc

SEQ ID NO: 24 - DNA, 45 bp, synthetic construct
  source 1..45: mol_type = other DNA
gcctttgaccagaggaacgacctgattcatcaggcgcctgttacg

SEQ ID NO: 25 - DNA, 19 bp, synthetic construct
  source 1..19: mol_type = other DNA
taatacgactcactatagg

SEQ ID NO: 26 - DNA, 19 bp, synthetic construct
  source 1..19: mol_type = other DNA
gctagttattgctcagcgg

SEQ ID NO: 27 - DNA, 24 bp, synthetic construct
  source 1..24: mol_type = other DNA
tagtattctctggattcacgcggt

SEQ ID NO: 28 - DNA, 24 bp, synthetic construct
  source 1..24: mol_type = other DNA
aaacaccgcgtgaatccagagaat

SEQ ID NO: 29 - DNA, 20 bp, synthetic construct
  source 1..20: mol_type = other DNA
gatgtgctggtttgtgacgg

SEQ ID NO: 30 - DNA, 20 bp, synthetic construct
  source 1..20: mol_type = other DNA
gacgcatcatccagaccgat

SEQ ID NO: 31 - DNA, 18 bp, synthetic construct
  source 1..18: mol_type = other DNA
agcgagtcagtgagcgag

SEQ ID NO: 32 - DNA, 22 bp, synthetic construct
  source 1..22: mol_type = other DNA
ccgccctcagaaaaactcatcg

SEQ ID NO: 33 - DNA, 42 bp, synthetic construct
  source 1..42: mol_type = other DNA
cgatgagtttttctgagggcggtccccctcaagtcaaaagcc

SEQ ID NO: 34 - DNA, 50 bp, synthetic construct
  source 1..50: mol_type = other DNA
ttcacactctagagggatccgtcaacgaattcaagcttgatatcattcag

SEQ ID NO: 35 - DNA, 33 bp, synthetic construct
  source 1..33: mol_type = other DNA
ggatccctctagagtgtgaaattgttatccgct

SEQ ID NO: 36 - DNA, 46 bp, synthetic construct
  source 1..46: mol_type = other DNA
aacaatttcacactctagaggttttcattgggaagccagaatgatt

SEQ ID NO: 37 - DNA, 40 bp, synthetic construct
  source 1..40: mol_type = other DNA
ggctcgtcctgaatgatatctcagtcgcggtacaggatgg

SEQ ID NO: 38 - DNA, 42 bp, synthetic construct
  source 1..42: mol_type = other DNA
aacaatttcacactctagagctttgaccagaggaacgaccgt

SEQ ID NO: 39 - DNA, 38 bp, synthetic construct
  source 1..38: mol_type = other DNA
ggctcgtcctgaatgatatctcagcgcttgcgcagaat

SEQ ID NO: 40 - DNA, 46 bp, synthetic construct
  source 1..46: mol_type = other DNA
aacaatttcacactctagagcgaaaagtgactgagcgtacatgtat

SEQ ID NO: 41 - DNA, 41 bp, synthetic construct
  source 1..41: mol_type = other DNA
ggctcgtcctgaatgatatcctagaaacgaaccagcgcgga

SEQ ID NO: 42 - DNA, 45 bp, synthetic construct
  source 1..45: mol_type = other DNA
aacaatttcacactctagaggtgacattctggttgcaggtatgag

SEQ ID NO: 43 - DNA, 42 bp, synthetic construct
  source 1..42: mol_type = other DNA
ggctcgtcctgaatgatatcctactcttcttcgttcccgccc

SEQ ID NO: 44 - DNA, 28 bp, synthetic construct
  source 1..28: mol_type = other DNA
ctttctgctatggaggtcaggtatgatt

SEQ ID NO: 45 - DNA, 21 bp, synthetic construct
  source 1..21: mol_type = other DNA
ttgtccagcagggttgtccac

SEQ ID NO: 46 - DNA, 21 bp, synthetic construct
  source 1..21: mol_type = other DNA
ctgcctcggtgagttttctcc

SEQ ID NO: 47 - DNA, 54 bp, synthetic construct
  source 1..54: mol_type = other DNA
gcaaattgcgtcatgttttaatccttatcctagtacgctcagtcacttttcggt

SEQ ID NO: 48 - DNA, 54 bp, synthetic construct
  source 1..54: mol_type = other DNA
accgaaaagtgactgagcgtactaggataaggattaaaacatgacgcaatttgc

SEQ ID NO: 49 - DNA, 27 bp, synthetic construct
  source 1..27: mol_type = other DNA
ctctagagtgtgaaattgttatccgct

SEQ ID NO: 50 - DNA, 24 bp, synthetic construct
  source 1..24: mol_type = other DNA
gatatcattcaggacgagcctcag

SEQ ID NO: 51 - DNA, 47 bp, synthetic construct
  source 1..47: mol_type = other DNA
aacaatttcacactctagaggatcaagaggacaaccgttaatgagca

SEQ ID NO: 52 - DNA, 41 bp, synthetic construct
  source 1..41: mol_type = other DNA
ggctcgtcctgaatgatatcctagcggtgcagaacgtactg

SEQ ID NO: 53 - DNA, 37 bp, synthetic construct
  source 1..37: mol_type = other DNA
agaaggagatatacatatgagcaccgatagccagtac

SEQ ID NO: 54 - DNA, 38 bp, synthetic construct
  source 1..38: mol_type = other DNA
agctcgaattcggatccctagcggtgcagaacgtactg

SEQ ID NO: 55 - DNA, 22 bp, synthetic construct
  source 1..22: mol_type = other DNA
gtacgctcagtcacttttcggt

SEQ ID NO: 56 - DNA, 29 bp, synthetic construct
  source 1..29: mol_type = other DNA
gataaggattaaaacatgacgcaatttgc

SEQ ID NO: 57 - DNA, 44 bp, synthetic construct
  source 1..44: mol_type = other DNA
accgaaaagtgactgagcgtacatgagcaccgatagccagtacc

SEQ ID NO: 58 - DNA, 50 bp, synthetic construct
  source 1..50: mol_type = other DNA
gcaaattgcgtcatgttttaatccttatcctagcggtgcagaacgtactg

SEQ ID NO: 59 - DNA, 40 bp, synthetic construct
  source 1..40: mol_type = other DNA
accatcatcaccacagccagagcactatcgaagaacgcgt

SEQ ID NO: 60 - DNA, 34 bp, synthetic construct
  source 1..34: mol_type = other DNA
aggcgcgccgagctcgttacgcctggtggccgtt

SEQ ID NO: 61 - DNA, 45 bp, synthetic construct
  source 1..45: mol_type = other DNA
ataagaaggagatatacatatggcaatattaggtttaggcacgga

SEQ ID NO: 62 - DNA, 48 bp, synthetic construct
  source 1..48: mol_type = other DNA
gcggtttctttaccagacttaactttcaataattaccgtggcacaagc

SEQ ID NO: 63 - DNA, 20 bp, synthetic construct
  source 1..20: mol_type = other DNA
gattatgcggccgtgtacaa

SEQ ID NO: 64 - DNA, 20 bp, synthetic construct
  source 1..20: mol_type = other DNA
ttgtacacggccgcataatc

SEQ ID NO: 65 - DNA, 20 bp, synthetic construct
  source 1..20: mol_type = other DNA
agaccacggtgagcaatgac

SEQ ID NO: 66 - DNA, 22 bp, synthetic construct
  source 1..22: mol_type = other DNA
attgcagtcctcttcggacagc

SEQ ID NO: 67 - DNA, 21 bp, synthetic construct
  source 1..21: mol_type = other DNA
tgagtacggtgggtttctggc

SEQ ID NO: 68 - DNA, 21 bp, synthetic construct
  source 1..21: mol_type = other DNA
tcaggaatacgcagcacctgc

SEQ ID NO: 69 - DNA, 18 bp, synthetic construct
  source 1..18: mol_type = other DNA
ccactgggccagcattgc

SEQ ID NO: 70 - DNA, 20 bp, synthetic construct
  source 1..20: mol_type = other DNA
gtaccaactgttcgtgcgcg

SEQ ID NO: 71 - DNA, 19 bp, synthetic construct
  source 1..19: mol_type = other DNA
gctgaccgtgcacatcagc

SEQ ID NO: 72 - DNA, 22 bp, synthetic construct
  source 1..22: mol_type = other DNA
cgtgttactcgtcggatttccg

SEQ ID NO: 73 - DNA, 46 bp, synthetic construct
  source 1..46: mol_type = other DNA
tctctagagtcgacctgcagaagcttgatgtgctggtttgtgacgg

SEQ ID NO: 74 - DNA, 46 bp, synthetic construct
  source 1..46: mol_type = other DNA
gataacagggtaatagatctaagcttgacgcatcatccagaccgat

SEQ ID NO: 75 - DNA, 21 bp, synthetic construct
  source 1..21: mol_type = other DNA
ctccaccgctgatgacatcag

SEQ ID NO: 76 - DNA, 53 bp, synthetic construct
  source 1..53: mol_type = other DNA
tgaagacttggatgctagcaagcttctttctgctatggaggtcaggtatgatt

SEQ ID NO: 77 - DNA, 46 bp, synthetic construct
  source 1..46: mol_type = other DNA
gggggcttttttattgcgcggatccttgtccagcagggttgtccac

SEQ ID NO: 78 - DNA, 20 bp, synthetic construct
  source 1..20: mol_type = other DNA
atgcctcagcggtgcttaaa

SEQ ID NO: 79 - DNA, 20 bp, synthetic construct
  source 1..20: mol_type = other DNA
cggagaaacgacctgacctt

SEQ ID NO: 80 - DNA, 22 bp, synthetic construct
  source 1..22: mol_type = other DNA
actggccgtcgttttacaacgt

SEQ ID NO: 81 - DNA, 27 bp, synthetic construct
  source 1..27: mol_type = other DNA
gtcatagctgtttcctgtgtgaggcgt

SEQ ID NO: 82 - DNA, 43 bp, synthetic construct
  source 1..43: mol_type = other DNA
ctagggtctcaactagtattatacctaggactgagctagctgt

SEQ ID NO: 83 - DNA, 50 bp, synthetic construct
  source 1..50: mol_type = other DNA
ctagggtctcagttttagagctagaaatagcaagttaaaataaggctagt

SEQ ID NO: 84 - DNA, 24 bp, synthetic construct
  source 1..24: mol_type = other DNA
tagtcggcggaacgattattcttg

SEQ ID NO: 85 - DNA, 24 bp, synthetic construct
  source 1..24: mol_type = other DNA
aaaccaagaataatcgttccgccg

SEQ ID NO: 86 - DNA, 20 bp, synthetic construct
  source 1..20: mol_type = other DNA
ccgtggagggagaagtgacg

SEQ ID NO: 87 - DNA, 23 bp, synthetic construct
  source 1..23: mol_type = other DNA
gctcatacctgcaaccagaatgt

SEQ ID NO: 88 - DNA, 41 bp, synthetic construct
  source 1..41: mol_type = other DNA
ttctggttgcaggtatgagctagctgatggagaggggcggt

SEQ ID NO: 89 - DNA, 21 bp, synthetic construct
  source 1..21: mol_type = other DNA
ccacggtttaagagcaaacat

SEQ ID NO: 90 - DNA, 36 bp, synthetic construct
  source 1..36: mol_type = other DNA
gccgcgcggcagccatatgtatacgaagattattgg

SEQ ID NO: 91 - DNA, 32 bp, synthetic construct
  source 1..32: mol_type = other DNA
gttagcagccggatccctagaaacgaaccagc

SEQ ID NO: 92 - DNA, 35 bp, synthetic construct
  source 1..35: mol_type = other DNA
gccgcgcggcagccatatgacgcaatttgcatttg

SEQ ID NO: 93 - DNA, 32 bp, synthetic construct
  source 1..32: mol_type = other DNA
gttagcagccggatccttaaagctcgagcgcc

SEQ ID NO: 94 - DNA, 35 bp, synthetic construct
  source 1..35: mol_type = other DNA
gccgcgcggcagccatatgattggcatcaaaagca

SEQ ID NO: 95 - DNA, 32 bp, synthetic construct
  source 1..32: mol_type = other DNA
gttagcagccggatcctcagtcgcggtacagg

SEQ ID NO: 96 - DNA, 35 bp, synthetic construct
  source 1..35: mol_type = other DNA
gccgcgcggcagccatatgcataacgtcgtgatca

SEQ ID NO: 97 - DNA, 29 bp, synthetic construct
  source 1..29: mol_type = other DNA
gttagcagccggatcctcagcgcttgcgc